SEQUENCE LISTING (13 sequences)

SEQ ID NO: 1 - DNA, 1451 bp, synthetic construct
  source 1..1451: mol_type = other DNA
atctgacttggttacgatggactttgaacacgccgagggtgactaaaccgctggatttacgcggttttcttctcttcaacttctttgacgtagcggtgaaccgtgcccaccgagcaaccaatctctgccgcgatagcgcgcatggacagacctttggcgcgcagctcctggacgcgagcacgcttctcgttggcacgtttaatgaacacttcacgcggttcggaagtccatcgttgagctgttctcaccgacataccggcacgttctgccagctcgcgggctgtttttccgttgcgtggataacgtgcataaaccttagccaatgttcctccaaagagtatgtccagcctcacgacgcacctcagcgcttcgttgccagcctttcttcccgcgtgcggattgcattgcggtgaatgtggcgtcgtagacggcggcgccgtctgtccacatgcgtgacttggtgatgatccatttatggattgacctggcaatacagtcaacttcggccacaggtagtggttcatcaaacagctcttggttaagtgcttgcgcggtggtttggattgcgcggcctaggccgtcagcgtctccaaaatgctttctgacctcccgatatgcccacgtacgtgcgctttcaaagagtgcgcaattacgacctagaccaactggcgagaaccgccgcgttttcctccaggacgcaggcggcataaagccggtttcttcgagccaaaagcgcagctcatcgagcgtatacagcttatcggtgatccagtgactatcccatgcagtgtgctcggggtttttggtgatcagcccggagtatccgctatcgccatcgacagagcgccgtaggccttcggtgacagccgcggcataggccaaaggcttgcgtttggcgtattcggtgcgggtaaatggctccgcgagcgcccagacagcgtgtgcgtgcccgtttaaggggttttcaaccaccgcgttaggtctccagtcctccctgtcccacaaagagcgcaaaagcgcgtcctcctggtcgatgtcaacgaccaggaggttagagagcgcgtcgggattggcttcgacgtagcgcttatccagcgcgttcttccgtgaggtgcggtaaatgccctcacggaggtcatcgcttgccaatggccacagcggcagccacagctgctcaaagcgtccctcagggcgggtagttggtctcatgtagctgactttctcacacgagcgtgcacggtcggttttcattcctcggcgtgttcataatacgacatttaaccaagtcagatgtttccccggtttccgggggttcccctgaagaacccttccagtgcgagcgaagcgagctcctttggccggcgcccctcaggtagccctctaaggctcccagggctccgcccctccctgaggttggctcaagcctcctggtggctcctacggacgttcta

SEQ ID NO: 2 - DNA, 1885 bp, synthetic construct
  source 1..1885: mol_type = other DNA
ggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatgct

SEQ ID NO: 3 - DNA, 4107 bp, synthetic construct
  source 1..4107: mol_type = other DNA
atggacaagaagtacagcatcggcctggacatcggcaccaacagcgtgggctgggcggtcatcaccgacgagtacaaggtcccctccaagaagttcaaggtcctgggcaacaccgaccggcactcgatcaagaagaacctgatcggcgccctgctcttcgacagcggcgaaaccgccgaggcgacccgcctgaagcggaccgcccgtcgccgctacacccggcgcaagaaccgcatctgctacctccaggagatcttctccaacgagatggccaaggtcgacgactcgttcttccaccggctcgaggagagcttcctggtggaggaggacaagaagcacgagcgccacccgatcttcggcaacatcgtcgacgaggtggcctaccacgagaagtaccccaccatctaccacctccgcaagaagctggtcgactcgaccgacaaggcggacctgcggctcatctacctggccctcgcgcacatgatcaagttccgcggccacttcctcatcgagggcgacctgaacccggacaactccgacgtcgacaagcttttcatccagctggtgcagacctacaaccagctgttcgaggagaaccccatcaacgccagcggcgtcgacgccaaggcgatcctctccgcgcgcctgagcaagtcccggcgcctggagaacctcatcgcccagctgccgggcgagaagaagaacggcctcttcggcaacctgatcgcgctgtcgctcggcctgacccccaacttcaagagcaacttcgacctggccgaggacgcgaagctccagctgtccaaggacacctacgacgacgacctggacaacctgctcgcccagatcggcgaccagtacgcggacctcttcctggccgcgaagaacctctcggacgccatcctgctcagcgacatcctgcgggtcaacaccgagatcaccaaggccccgctgtcggcgagcatgatcaagcggtacgacgagcaccaccaggacctgaccctgctcaaggccctcgtgcgccagcagctgcccgagaagtacaaggagatcttcttcgaccagtccaagaacggctacgccggctacatcgacggcggcgcgtcgcaggaggagttctacaagttcatcaagccgatcctggagaagatggacggcaccgaggagctgctcgtcaagctgaaccgcgaggacctgctccgcaagcagcggaccttcgacaacggctccatcccgcaccagatccacctgggcgagctccacgccatcctccggcgccaggaggacttctaccccttcctgaaggacaaccgcgagaagatcgagaagatcctgaccttccggatcccgtactacgtcggccccctggcccgcggcaactcccggttcgcgtggatgacccggaagtcggaggaaaccatcaccccgtggaacttcgaggaggtcgtggacaagggcgcctccgcgcagtcgttcatcgagcgcatgaccaacttcgacaagaacctcccgaacgagaaggtcctgcccaagcacagcctgctctacgagtacttcaccgtgtacaacgagctgaccaaggtcaagtacgtgaccgagggcatgcggaagccggccttcctgtccggcgagcagaagaaggcgatcgtcgacctgctcttcaagaccaaccgcaaggtcaccgtgaagcagctgaaggaggactacttcaagaagatcgagtgcttcgactccgtcgagatctcgggcgtggaggaccgcttcaacgcctccctgggcacctaccacgacctgctcaagatcatcaaggacaaggacttcctcgacaacgaggagaacgaggacatcctggaggacatcgtcctcaccctgaccctcttcgaggaccgcgagatgatcgaggagcggctcaagacctacgcccacctgttcgacgacaaggtgatgaagcagctgaagcggcgccggtacaccggctggggccgcctctcccggaagctgatcaacggcatccgggacaagcagagcggcaagaccatcctggacttcctcaagtccgacggcttcgccaaccgcaacttcatgcagctcatccacgacgactcgctgaccttcaaggaggacatccagaaggcccaggtgtccggccagggcgacagcctccacgagcacatcgccaacctggcgggctccccggcgatcaagaagggcatcctccagaccgtcaaggtcgtggacgagctggtcaaggtgatgggccgccacaagcccgagaacatcgtgatcgagatggcccgggagaaccagaccacccagaagggccagaagaactcccgcgagcggatgaagcgcatcgaggagggcatcaaggagctcggctcgcagatcctgaaggagcacccggtcgagaacacccagctccagaacgagaagctgtacctctactacctgcagaacggccgcgacatgtacgtggaccaggagctcgacatcaaccggctgagcgactacgacgtcgaccacatcgtgccgcagtccttcctgaaggacgactcgatcgacaacaaggtcctgacccgctccgacaagaaccggggcaagtccgacaacgtgccctcggaggaggtcgtgaagaagatgaagaactactggcgccagctgctcaacgccaagctcatcacccagcgcaagttcgacaacctgaccaaggccgagcggggcggcctgtcggagctcgacaaggcgggcttcatcaagcgccagctcgtcgaaacccggcagatcaccaagcacgtggcccagatcctggacagccggatgaacaccaagtacgacgagaacgacaagctgatccgcgaggtcaaggtgatcaccctcaagagcaagctggtgtccgacttccgcaaggacttccagttctacaaggtccgggagatcaacaactaccaccacgcccacgacgcgtacctgaacgccgtcgtgggcaccgcgctgatcaagaagtacccgaagctggagtccgagttcgtctacggcgactacaaggtctacgacgtgcgcaagatgatcgccaagtcggagcaggagatcggcaaggccaccgcgaagtacttcttctacagcaacatcatgaacttcttcaagaccgagatcaccctggccaacggcgagatccgcaagcggcccctgatcgaaaccaacggcgaaaccggcgagatcgtctgggacaagggccgcgacttcgccaccgtccggaaggtgctgtccatgccgcaggtcaacatcgtcaagaaaaccgaggtgcagaccggcggcttcagcaaggagtccatcctccccaagcgcaactcggacaagctgatcgcccggaagaaggactgggacccgaagaagtacggcggcttcgacagccccaccgtcgcctactccgtgctggtcgtggcgaaggtcgagaagggcaagagcaagaagctgaagtccgtgaaggagctgctcggcatcaccatcatggagcgctcctcgttcgagaagaacccgatcgacttcctggaggccaagggctacaaggaggtcaagaaggacctcatcatcaagctgcccaagtactcgctgttcgagctcgagaacggccgcaagcggatgctcgccagcgcgggcgagctgcagaagggcaacgagctggccctcccgtccaagtacgtcaacttcctgtacctcgcgtcccactacgagaagctgaagggctcgcccgaggacaacgagcagaagcagcttttcgtggagcagcacaagcactacctggacgagatcatcgagcagatctcggagttcagcaagcgggtcatcctggccgacgcgaacctcgacaaggtgctgtccgcctacaacaagcaccgcgacaagccgatccgggagcaggcggagaacatcatccacctgttcaccctcaccaacctgggcgcccccgccgcgttcaagtacttcgacaccaccatcgaccgcaagcggtacaccagcaccaaggaggtcctcgacgcgaccctgatccaccagtccatcaccggcctgtacgaaacccgcatcgacctctcccagctcggcggcgactga

SEQ ID NO: 4 - DNA, 59 bp, synthetic construct
  source 1..59: mol_type = other DNA
cgtcaagatcacccaaaactggtggctgttctcttttaagcgggatagcatgggttctt

SEQ ID NO: 5 - DNA, 97 bp, synthetic construct
  source 1..97: mol_type = other DNA
tgccgtttctcgcgttgtgtgtggtactacgtggggacctaagcgtgtaagatggaaacgtctgtatcggataagtagcgaggagtgttcgttaaaa

SEQ ID NO: 6 - DNA, 92 bp, synthetic construct
  source 1..92: mol_type = other DNA
taactacattgagcgaaatgccaaccacatgtcccatgcttttactaatgtggggtcttagaagaaagcgaccaatttaaggagagttgaat

SEQ ID NO: 7 - DNA, 49 bp, synthetic construct
  source 1..49: mol_type = other DNA
agtgaacccatacttttatatatgggtatcggcggtctatgcttgtggg

SEQ ID NO: 8 - DNA, 74 bp, synthetic construct
  source 1..74: mol_type = other DNA
tccctatcagtgatagagattgacatccctatcagtgatagagatactgagcacatcagcaggacgcactgacc

SEQ ID NO: 9 - DNA, 80 bp, synthetic construct
  source 1..80: mol_type = other DNA
tcgtcaagatcacccaaaactggtggctgttctcttttaagcgggatagcatgggttcttatccctatcagtgatagaga

SEQ ID NO: 10 - DNA, 62 bp, synthetic construct
  source 1..62: mol_type = other DNA
ttgacaattaatcatcggctcgtataatgtgtggaattgtgagcggataacaatttcacaca

SEQ ID NO: 11 - DNA, 101 bp, synthetic construct
  source 1..101: mol_type = other DNA
ctcgagggtaaatgtgagcactcacaattcattttgcaaaagttgttgactttatctacaaggtgtggcataatgtgtgtaattgtgagcggataacaatt

SEQ ID NO: 12 - DNA, 354 bp, synthetic construct
  source 1..354: mol_type = other DNA
acttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaa

SEQ ID NO: 13 - DNA, 226 bp, synthetic construct
  source 1..226: mol_type = other DNA
gagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaacc

(19) United States
(12) Patent Application Publication
(10) Pub. No.: US 2023/0074594 A1
(43) Pub. Date: Mar. 9, 2023

(54) GENOME EDITING USING CRISPR IN CORYNEBACTERIUM

(71) Applicant: Zymergen Inc., Emeryville, CA (US)

(72) Inventors: Stephen BLASKOWSKI, Oakland, CA (US); Robert COATES, Oakland, CA (US); Kedar PATEL, Fremont, CA (US); Hendrik Marinus VAN ROSSUM, Oakland, CA (US); Shawn SZYJKA, Martinez, CA (US)

(21) Appl. No.: 17/817,223

(22) Filed: Aug. 3, 2022

Related U.S. Application Data
(63) Continuation of application No. 16/943,761, filed on Jul. 30, 2020, now abandoned.
Application No. 16/943,761 is a continuation of application No. PCT/US2019/017276, filed on Feb. 8, 2019.
(60) Provisional application No. 62/701,979, filed on Jul. 23, 2018.
(60) Provisional application No. 62/628,166, filed on Feb. 8, 2018.

(51) Int. Cl. C12N 15/113; C12N 9/22; C12N 15/77

(52) U.S. Cl. CPC C12N 15/113; C12N 9/22; C12N 15/77; C12N 2310/20; C12N 1/205

(57) ABSTRACT

A CRISPR system is successfully used to modify the genomes of a gram-positive bacterium, such as a species of the Cornybacterium genus. Methods for modifying Corynebacterium species include single-nucleotide changes, creating gene deletions and/or insertions.

### CROSS-REFERENCE TO RELATED APPLICATIONS

This application is a continuation of U.S. application Ser. No. 16943,761, filed Jul. 30, 2020, which is a continuation of International PCT Application No. PCT/US2019/017276, filed Feb. 8, 2019, which claims the benefit of U.S. Provisional Application No. 62/628,166, filed Feb. 8, 2018, and U.S. Provisional Application No. 62/701,979, filed Jul. 23, 2018, the contents of each of which are hereby incorporated by reference in their entirety.

### STATEMENT REGARDING FEDERALLY SPONSORED RESEARCII OR DEVELOPMENT

The present disclosure was made with Government support under Contract No. HR0011-15-9-0014, awarded by the U.S. Government Defense Advanced Research Projects Agency (DARPA). The Government has certain rights in the invention.

### REFERENCE TO AN ELECTRONIC SEQUENCE LISTING

The contents of the electronic sequence listing (ZYMR_038_03US_SeqUst_ST25.xml; Size: 20,884 bytes: and Date of Creation: Aug. 3, 2022) is herein incorporated by reference in its entirety,

### FIELD OF INVENTION

The present disclosure generally relates to systems, methods, and compositions used for guided genetic sequence editing in vivo and in vitro using CRISPR-mediated editing to modify the genome of the gram-positive bactetium Corynebacterium glutamicum (C. glutamicum). The disclosure describes, inter alia, methods of using guided sequence editing complexes for improved DNA cloning, assembly of oligonucleotides, and for the improvement of microorganisms.

### BACKGROUND OF THE INVENTION

The ability to modify genomes and improve microbes has increased significantly over the past several decades. Early approaches involved UV or chemical mutagenesis and transposons (Karberg et al., 2001; Perutka et al., 2004; Yao and Lambowitz, 2007), typically resulting in functional knockouts and small alterations (e.g. single nucleotide polymorphisms or SNPs). Site specific integration techniques involving integrases and recombinases enabled insertion of genes and pathways, but were confined to specific pre-existing sequences or “landing pads” in genomes (Kilby et al., 1993; Sternberg et at, 1981; Turan et al., 2011).

The ability to harness homologous recombination (HR) to precisely engineer a genomic region of interest to create SNPs, deletions and insertions has enabled great sttides in all areas of biotechnology (therapeutics, production of novel chemicals, metabolic engineering etc). Early technologies introduced double-stranded DNA as well as single stranded oligonucleotide donor DNA into the host genome by harnessing endogenous host homologous recombination machinery or by supplementing with viral recombination proteins (Datsenko and Wanner, 2000; van Pijkeren and Britton, 2012; Yu et al., 2000; Zhang et at., 1998). Unfortunately, however, these methods rely on low frequency events and require the use of a selection marker to select for the change being introduced, which must then be removed prior to each round of engineering in order to incorporate multiple mutations.

It was subsequently discovered that creating a double strand break (DSB) at or near the site being engineered could significantly increase the frequency of recombination events at that site. In recombination-proficient organisms such as Saccharomyces cerevisiae, for example, a double stranded break induces the homologous recombination machinery, which increases the efficiency of subsequent DNA incorporation (Symington, 2002). This was initially exploited by pre-installing “landing pads” with meganucleases (e.g. I-SceI and I-CeuI), inducing DSBs by expressing the appropriate horning endonuclease, and supplying an appropriate donor nucleic acid with homologous ends flanking the changes being introduced (Kuhlman and Cox, 2010). Later, technologies harnessed binding domains fused to nuclease domains (e.g. Zinc Finger Nucleases or ZFNs and Transcription Activator-Like Effector Nucleases or TALENs, which are binding domains fused to the Foki nuclease) which allowed for programmable marked ess editing by enabling the creation of double strand breaks at any given genomic location (Hockerneyer et at, 2011; Li et at , 2011; Miller et al., 2011; Urnov et al., 2005).

More recently, a new class of RNA-guided endonucleases have been identified in some bacteria and archaea relying on Clustered Regularly Interspaced Short Palindromic Repeat (CRISPR) genomic loci and CIUSPR-associated (Cas) proteins, that function together to provide protection from invading viruses and plasmids. In Type II CRISPR-Cas systems, the Cas9 protein functions as an RNA-guided endonuclease that uses a dual-guide RNA consisting of CRISPR RNA (crRNA) and trans-activating crRNA (tracrRNA) for target recognition and cleavage by a mechanism involving two nuclease active sites that together generate double-stranded DNA breaks (DSBs). Jinek et al. later showed that crRNA and tracrRNA can be cotnbi tied and transcribed as a single guide RNA (sgRNA), wherein a short 20-nt ‘spacer’ sequence within the sgRNA directs the Cas9 protein to its DNA target (Jinek et al., 2012).

Functionally, the Cas9 protein recognizes and complexes with the guide RNA, and the resulting ribonucleoprotein complex then searches the genome for a sequence complementary to the spacer sequence (the ‘proto-spacer’) followed by the proto-spacer adjacent motif (PAM). Under one model, upon binding to the target sequence, Cas9 creates a double stranded break, and changes can then be introduced either by non-homologous end joining (NHEJ) or by HR of supplied donor nucleic acid. NHEJ results in short insertions or deletions, which can be useful in creating functional knockouts by frameshift mutations in open reading frames (OREs). HR, in contrast, can be utilized to introduce more precise seamless changes in genomes by incorporation of appropriately designed donor nucleic acid.

However, in hosts having limited or no endogenous machinery for homologous recombination, a Cas9-targeted double strand break in the genome can often result in toxicity and, cell death. In these organisms, alternative strategies have been developed for inducing single strand nicks, as these breaks are not lethal. In Clostridium cellulolyticum, for example, expression of the wild-type Cas9 protein is lethal, which was circumvented in a study by Xu et al. (2015) via. expression of the Cas9^{D10A} nickase to produce a nick in one DNA strand in the genome, which then served as a site for increased FIR with a supplied donor DNA. Although gene deletions could be obtained with reasonable efficiency following this methodology, there were significant limits on the integration of larger (e.g. >1 kb) inserts.

C. glutamicum is another prokaryote having limited endogenous machinery for HR (Resende et al., Gene. 2011;482:1-7;1-7; Nakamura et al., Gene. 2003 Oct. 23; 317(1-2):149-55), and hence an uncertain outcome with respect to CRISPR/Cas9-mediated gene editing. There are currently several publications that describe the use of CRISPR1Cas9 in C. glutamicum (Jiang et al. Nat Commun, 2017 May 4;8:15179; Cho et al. Metab Eng. 2017 July; 42:157-167; Peng et at. Microb Cell Fact.; 2017 Nov 14;16(1):201; Liu et al. Microb Cell Fact. 2017 Nov. 16; 16(1):205). These publications test several editing configurations and C. glutamicum strains. Interestingly, however, the publications vary widely in their conclusions regarding Cas9 toxicity, efficacy of editing, specific strains tested, as well as donor configurations tested. Moreover, with respect to multiplex gene editing in particular, the C. glutamicum art has focused exclusively on simultaneous introduction of multiple edits, but has achieved only very low editing efficiencies of continuous polynucleotide regions. For example, Liu et al. describes simultaneous editing of a first (rfp) and a second (rpsL) locus, using an rfp-directed and an rpsL-directed guide RNA respectively. However, only very low editing efficiencies were achieved and only continuous regions were edited within each locus. Thus, there remains a need for improved RNA-guided endonuclease tools and methods for genome editing in C. glutamicum, including improved multiplex gene editing methods that provide greater efficiency, non-contiguous editing within one or more loci, and other benefits.

### SUMMARY OF THE INVENTION

The present invention addresses these unmet needs and uncertainties with successful methods for genetically modifying Corynebacterium using a CRISPR system comprising a RNA-guided endonuclease (e.g., Cas9) polypeptide, a guide RNA, and a donor polynucleotide, wherein the donor polynucleotide is preferably encoded in a replicating plasmid. In particular embodiments, methods for multiplex gene modification with improved editing efficiency are exemplified employing simultaneous or sequential, plasmid-based presentation of two or more donor polynucleotides. Certain embodiments of the present invention are described in Coates, et al., “Systematic investigation of CRISPR-Cas9 configurations for flexible and efficient genome editing in Corynebacterium glutamicum NRRL-B11474”, J Ind Biotechnol, published online 2018 Nov. 27 (doi: 10.1007/s10295-018-2112-7), which is hereby incorporated by reference in its entirety.

In certain embodiments, methods for introduction of multiple edits with improved editing efficiency are exemplified employing plasmid-based presentation of one, or more, donor polynucleotide(s) encoding two or more genome modifications. In some cases, the donor polynucleotide(s) (whether provided by plasmid-based presentation or as a linear fragment) encode two or more genome modifications, where at least two of the two or more genome modifications, or all of the two or more modifications, are, e.g., non-contiguous and, in close proximity to one another in the genome. In some cases, the modifications that are in close proximity to one another in the genome are within, or within about, 150 base pairs, 125 base pairs, 100 base pairs, 75 base pairs, 70 base pairs, 65 base pairs, 60 base pairs, 55 base pairs, 50 base pairs, 45 base pairs, 40 base pairs, 35 base pairs, 30 base pairs, 25 base pairs, 20 base pairs, or 10 or 5 base pairs. In some cases, the modifications that are in close proximity to one another in the genome are at a distance from each other in the genome of from about 10 to about 100 base pairs, or from about 25 to about 75 base pairs.

Moreover, many of the recent publications directed to the use of CRISPR/Cas9 in C. glutamicum utilize plasmids having a pB1,1 replication origin. During the course of the work described herein, it was determined that pail and pCASEI plasmids were unexpectedly superior to the pBL1 -based plasmids in the test strain NRRL-B11474. Without wishing to be bound by theory, the present inventors hypothesize that the unexpectedly improved transformation efficacy of pCG1 and pCASE1-based plasmids in comparison to pB-1-based plasmids is applicable to a wide range of commercially relevant Cognebacterium strains, including other C. glutamicum strains. Again, without wishing to be bound by theory, it is further hypothesized that the unexpectedly improved editing is at least in part due to an unexpected increase in transformation efficiency Obtained using pCG1 and pCASE1 plasmids in comparison to pBL1-based plasmids. Accordingly, also described herein is the construction of a more effective RNA-guided endonuclease (e.g, Cash) -editing system and an expanded toolbox of possible donor configurations, including but not limited to donor configurations using pCG1 and/or pCASE1 plasmid-encoding of one or more donor polynucleotides.

In one aspect, the invention provides methods for genetically modifying a Corynebacterium host comprising transforming a Corynebacterium host with a plasmid comprising a first promoter, e.g., an inducible or a constitutive promoter, operably linked to a sequence for expressing a first guide RNA, and providing a first donor polynucleotide having a left homology arm sequence and a right homology arm sequence each homologous to a Corynebacterium target sequence, and expressing said first guide RNA in conjunction with a RNA-guided endonuclease (e.g., Cas9)polypeptide in the host. In some cases, said donor polynucleotide is encoded on a plasmid.

In one aspect, the invention provides methods for genetically modifying a. Corynebacterium host comprising transforming a Corynebacterium species with a first plasmid comprising a first promoter operably linked to a sequence for expressing an RNA-guided DNA endonuclease polypeptide, providing a first guide RNA, and expressing said RNA-guided DNA endonucl ease polypeptide in conjunction with a first donor polynucleotide having a left homology arm sequence and a right homology arm sequence each homologous to a Corynebacterium target sequence in said host, said first donor polynucleotide including at least one mutation sequence flanked by said left and right homology arm sequences.

In some embodiments, the RNA-guided DNA endonuclease is selected from the group consisting of Cas9, Cas12a, Cas12b, Cas12c, Cas12d, Cas12e, Cas12h, Cas13a, Cas13b, Cas13c, Cpf1, and MAD7, or homologs, orthologs, or paralogs thereof. In some embodiments, the RNA-guided DNA endonuclease is Cas9.

In some embodiments, the first donor polynucleotide is provided by plasmid-based presentation. In some embodiments, the first guide RNA is provided by plasmid-based presentation. In some embodiments, the RNA-guided DNA endonuclease is provided by plasmid-based presentation. In some embodiments, the RNA-guided DNA endonuclease is integrated into the genome of said Corynebacterium species.

In some embodiments, the Corynebacterium species is Corynebacterium glutamicum. In some embodiments, the Corynebacterium species is Corynebacterium glutamicum strain NRRL-B11474.

In some embodiments, the first plasmid comprises a replication origin selected from the group consisting of a pCASE1 replication origin and a pCGi replication origin. In some embodiments, the first plasmid comprises a pCASE1 replication origin. In some embodiments, the first plasmid comprises a pCG1 replication origin.

In some embodiments, the first donor polynucleotide is provided on the same plasmid as the guide RNA. In some embodiments, the first donor polynucleotide is provided on a different, second or third, plasmid. In some embodiments, the first donor polynucleotide is provided as a linear, single- or double-stranded DNA fragment. In some embodiments, a plasmid or linear or circular fragment comprising or encoding the first donor polynucleotide further comprises or encodes for one or more, or two or more, additional donor polynucleotides.

In some embodiments, said first donor polynucleotide is provided on said first plasmid. In some embodiments, said first donor polynucleotide is provided on a second plasmid. In some embodiments, said first donor polynucleotide is provided as a linear fragment. In some embodiments, said first plasmid further encodes the first guide RNA or one or more additional guide RNAs operably linked to said first promoter, or one or more additional guide RNAs operably linked to a second promoter. In some embodiments, said first promoter is constitutive. In some embodiments, said first promoter is inducible. In some embodiments, said second promoter is constitutive. In some embodiments, said second promoter is inducible. In some embodiments, said first promoter and said second promoter are inducible. In some embodiments,said first promoter and said second promoter are differentially inducible. In some embodiments, said first donor polynucleotide is provided on said first plasmid and said first plasmid further comprises one or more additional donor polynucleotide(s) having left homology arm sequence(s) and a right homology arm sequence(s) each homologous to a Corynebacterium target sequence, said additional donor polynucleotide(s) each including at least one mutation sequence flanked by said left and right homology arm sequences. In some embodiments, said first donor polynucleotide is provided on said second plasmid and said wherein said second plasmid further comprises one or more additional donor polynucleotide(s) having left homology arm sequences) and a right homology arm sequence(s) each homologous to a Cognebacierium target sequence, said additional donor polynucleotide(s) each including at least one mutation sequence flanked by said left and right homology arm sequences. In some embodiments, the first plasmid or the second plasmid encodes the RNA-guided. DNA endonuclease.

In certain embodiments, said first donor polynucleotide includes at least one mutation sequence flanked by said left homology arm sequence and said right homology arm sequence. The at least one mutation sequence may advantageously comprise a mutation selected from the group consisting of: a single nucleotide insertion; an insertion of two or more nucleotides; an insertion of a nucleic acid sequence encoding one or more proteins; a single nucleotide deletion; a deletion of two or more nucleotides; a deletion of one or more coding sequences; a substitution of a single nucleotide; and a substitution of two or more nucleotides. In specific embodiments, the at least one mutation sequence comprises a mutation of a Cas9 PAM or seed region. In certain embodiments, said at least one mutation sequence comprises multiple mutations according to the previous sentence. In certain embodiments, the multiple mutations are encoded on the same donor polynucleotide sequence.

In some embodiments, the at least one mutation sequence comprises a mutation of an RNA-guided DNA endonuclease protospacer-adiacent motif (RAM) or seed region. In some embodiments, the Corynebacterium host further expresses a functional RNA-guided DNA endonuclease polypeptide encoding sequence linked to a constitutive or an inducible promoter. In some embodiments, the first plasmid further comprises a fiinctional RNA-guided DNA endonuclease polypeptide encoding sequence operably linked to a constitutive or an inducible promoter. In some embodiments, the first plasmid further comprises a functional Cas9 polypeptide encoding sequence operably linked to a constitutive or an inducible promoter.

In some embodiments, the Corynebacterium host comprises a sequence linked to a. constitutive or an inducible promoter, wherein the sequence encodes the RNA-guided DNA endonuclease polypeptide. In some embodiments, the first plasmid comprises a sequence operably linked to a constitutive or an inducible promoter, wherein the sequence encodes the RNA-guided DNA endonuclease polypeptide. In some embodiments, the RNA-guided DNA endonuclease polypeptide is a Cas9 endonuclease polypeptide.

In some embodiments, the left and/or right homology arm sequences on donor polynucleotides are least 25 base pairs, preferably at least 75 base pairs, more preferably at least 150, 200, 250, or 300 base pairs, still more preferably at least 350, 400, 450, 500, or 2,000 base pairs. In some embodiments, the promoter is selected from the group consisting of endogenous, heterologous, synthetic, inducible, and/or constitutive promoters. In some embodiments, the promoter is Pcg2613. In some embodiments, the first guide RNA comprises a CRISPR RNA (crRNA) and a trans-activating crRNA (tracrRNA). In some embodiments, the first guide RNA comprises a single gRNA (sgRNA). In some embodiments, the first guide RNA comprises a CRISPR RNA (crRNA) and a trans-activating RNA (tracrRNA) and a first spacer sequence of at least 20 nucleotides wherein said first spacer sequence is at least 80%, 85%, 90%, 95%, or 100% complementary to said Corynebacterium target sequence, in some embodiments, the method comprises sequentially inducing expression of two or more different guide RNAs and thereby introducing two or more different genetic modifications of the Corynebacterium host. In some embodiments, at least one of the two or more different genetic modifications comprise non-contiguous insertions, deletions, and/or substitutions; or wherein two or more of the two or more different genetic modifications each comprise non-contiguous insertions, deletions, and/or substitutions. In some embodiments, the method comprises sequentially expressing two or more different guide RNA/donor polynucleotide pairs under the control of different inducible promoters and thereby sequentially introducing two or more different genetic modifications of the Cotynebacteriwn host, wherein successive edits are introduced by serially inducing the expression of each successive guide RNA/donor polynucleotide pair. In some embodiments, the method comprises expressing two or more donor polynucleotides in the Corynebacterium host and sequentially providing gRNA(s) corresponding to the already present repair fragments in the host, thereby sequentially introducing two or more different genetic modifications of the Corynebacterium host. In some embodiments, the method comprises simultaneously expressing two or more different guide RNAs and thereby introducing two or more different genetic modifications of the Corynebacterium host. In some embodiments, the first plasmid comprises a counterselection marker, and the method comprises selecting against the counterselection marker and thereby curing the Corynebacterium host of the first plasmid. In some embodiments, the selecting against the counterselection marker of the first plasmid is performed after genetically modifying the Corynebacterium host with the first guide RNA in conjunction with the RNA-guided DNA endonuclease polypeptide in said host and before genetically modifying the Corynebacterium host with a second guide RNA in conjunction with the RNA-guided DNA endonuclease polypeptide in said host. In some embodiments, the at least one of the two or more different genetic modifications comprise non-contiguous insertions, deletions, and/or substitutions; or wherein two or more of the two or more different genetic modifications each comprise non-contiguous insertions, deletions, and/or substitutions. In some embodiments, the method comprises expressing a set of proteins from one or more heterologous recombination systems in said host. In some embodiments, the method comprises expressing a set of proteins from a lambda red recombination system, a Rec ET recombination system, any homologs, orthologs or paralogy of proteins from a lambda red recombination system or a Rec ET recombination system, or any combination thereof. In certain embodiments, the Corynebacterium host further comprises a functional Cas9 polypeptide encoding sequence operably linked to a constitutive or inducible promoter. In some embodiments, the plasmid further expresses the functional Cas9 polypeptide encoding sequence linked to a constitutive or inducible promoter. In some cases, the Cas9 promoter is differentially inducible as compared to a constitutive or an inducible promoter operably linked to a guide-RNA. In certain embodiments, said first plasmid further comprises a functional Cas9 polypeptide encoding sequence operably linked to a constitutive or an inducible promoter.

In some embodiments, the functional Cas9 polypeptide encoding sequence linked to a constitutive or inducible promoter is integrated into the host genome. Prior attempts to transform replicating plasmids with Cas9 have been reported to yield few transformants or exhibit reduced growth rates, potentially due to the toxicity of the Cas9 gene and/or polypeptide to the cell (Cho et al. Metab Eng. 2017 July; 42:157-167; Peng et al. Microb Cell Fact.; 2017 Nov. 14;16(I):201). As exemplified herein for the first time, the present inventors have determined that functional Cas9 polypeptides can be successfully integrated into the Corynebacterium genome without toxicity to the host, and without the need for reducing the toxicity of Cas9 itself. In some cases the toxicity of the Cas9 polypeptide, whether encoded by a plasmid or encoded by a gene integrated into the genome, is reduced by using an inducible promoter such that the Cas9 polypeptide is not expressed. or minimally expressed in a non-induced state and then expressed in an induced state to perform genome editing. In some cases, the Cas9 promoter is differentially inducible as compared to an inducible promoter operably linked to a guide-RNA.

In some embodiments, the functional Cas9 polypeptide encoding sequence linked to a constitutive or inducible promoter is in a plasmid. In some cases, the functional Cas9 polypeptide encoding sequence linked to a constitutive or inducible promoter is in the first plasmid. In some cases, the functional Cas9 polypeptide encoding sequence linked to a constitutive or inducible promoter is in a second plasmid. In some cases, the functional Cas9 polypeptide encoding sequence linked to a constitutive or inducible promoter is in a plasmid comprising a guide-RNA encoding sequence, e.g., the first guide-RNA encoding sequence. In some cases, the functional Cas9 polypeptide encoding sequence linked to a constitutive or inducible promoter is in a plasmid comprising a donor polynucleotide, e.g., the first donor polynucleotide. In some embodiments, a. plasmid comprising the functional Cas9 polypeptide encoding sequence linked to a constitutive or inducible promoter further comprises a counterselection marker. In some cases, said counterselection marker in a plasmid comprising Cas9 polypeptide encoding sequence can be differentially counterselected. In comparison to another counterselection marker on a different (e.g., first or second) plasmid. In some cases, the method comprises, after performing one or more, or two or more preferably sequential genomic edits of the Corynebacterium host, counterselecting against a plasmid comprising the functional Cas9 polypeptide encoding sequence, and thereby curing the Corynebacterium host of the Cas9 polypeptide encoding sequence.

In some embodiments, the guide RNA is a single-molecule guide RNA (sgRN.A). In some embodiments, the guide RNA is a dual-molecule guide RNA, e.g., crRNA and tracrRNA. In some embodiments, the first plasmid further encodes the first guide RNA or one or more additional guide RNAs operably linked to said first promoter, or one or more additional guide RNAs operably linked to a second promoter. In some cases, said first promoter is constitutive. In some cases, said first promoter is inducible. In some cases, the second promoter is constitutive. In some cases, the second promoter is inducible. In some cases, said first promoter and the second promoter are inducible. In some cases, said first promoter and the second promoter are differentially inducible. In some cases, said first promoter operably linked to said first guide RNA is induced, the genome of the Corynebacterium host is modified, and then the second promoter is induced and a different genetic modification of the Corynebacterium host is made. In some embodiments, the right and left homology arm sequences each independently comprises, comprises about, comprises at least, or comprises at least about, 25; 50; 75; 100; 125; 150; 175; 200; 225; 250; 275; 300; 325; 350; 375; 400; 425; 450; 475; 500; 525; 550; 575; 600; 625; 650; 675; 700; 725; 750; 775; 800; 825; 850; 875; 900; 925; 950; 975; 1,000; 1,025; 1,050; 1,075; 1,100; 1,125; 1,150; 1,175; 1,200; 1,225; 1,250; 1,275; 1,300; 1,325; 1,350; 1,375; 1,400; 1,425; 1,450; 1,475; 1,500; 1;525; 1,550; 1,575; 1,600; 1,625; 1,650; 1,675; 1,700; 1,725; 1,750; 1,775; 1,800; 1,825; 1,850; 1,875; 1,900; 1,925; 1,950; or 2,000 base pairs.

In certain embodiments, the right and left homology arm sequences each independently comprise between about 25 and about 2000 base pairs, between about 25 and about 1000 base pairs, between about 25 and about 600 base pairs, between about 25 and about 500 base pairs, between about 25 and about 250 base pairs, between about 25 and about 200 base pairs, between about 25 and about 100 base pairs, or between about 25 and about 50 base pairs. In certain embodiments, the right and left homology arm sequences each independently comprise between about 100 and about 2000 base pairs, between about 100 and about 1000 base pairs, between about 100 and about 600 base pairs, between about 100 and about 500 base pairs, between about 100 and about 250 base pairs, between about 100 and about 200 base pairs, or between about 100 and about 150 base pairs. In certain embodiments, the right and left homology arm sequences each independently comprise between about 0 and about 2000 base pairs, between about 0 and about 1000 base pairs, between about 0 and about 600 base pairs, between about 0 and about 500 base pairs, between about 0 and about 250 base pairs, between about 0 and about 200 base pairs, between about 0 and about 100 base pairs, between about 0 and about 50 base pairs, or between about 0 and about 25 base pairs.

In some embodiments, the promoter operably linked to the guide RNA is a native Corynebacterium promoter. In some embodiments, the promoter operably linked to the guide RNA is a promoter that is heterologous to the host cell. Generally, the promoter is heterologous to the operably linked guide RNA, whether the promoter is native to the host cell, derived from a different organism, or synthetic. In some embodiments, the promoter operably linked to the guide RNA is a synthetic promoter. The native, heterologous, or synthetic promoter can be constitutive. Alternatively, the native, heterologous, or synthetic promoter can be inducible. In certain embodiments, the promoter is selected from the group consisting of: Peg2613 or Pcg0007 or Pcg0047 or Pcg1133 or PTet1 or PTet3 or PLac1 or PLac2 or PAra1 or PTrc In certain embodiments, the promoter is Pcg2613. In certain embodiments, the promoter is selected from the group consisting of any endogenous Corynebacterium promoter, any promoter from a. heterologous organism, any synthetic promoter, any inducible promoter, or any constitutive promoter.

In some embodiments, the guide RNA is encoded by a first plasmid that comprises a counterselection marker, and the method comprises selecting against the counterselection marker and thereby curing the Corynebacterium host of the first plasmid. In some cases, the selecting against the counterselection marker of the first plasmid is performed after genetically modifying the Corynebacterium host with the first guide RNA in conjunction with the Cas9 polypeptide in said host and before genetically modifying the Corynebacterium host with a second guide RNA in conjunction with the Cas9 polypeptide in said host.

In another aspect, improved methods of multiplex gene editing are provided, comprising genetically modifying a Corynebacterium host with a first guide RNA expressed from a first plasmid in conjunction with a Cas9 polypeptide, selecting against a counterselection marker present on the first plasmid and thereby curing the host of the first plasmid, genetically modifying the Corynebacterium host with a second guide RNA expressed from a second plasmid in conjunction with a Cas9 polypeptide, selecting against a counterselection marker present on the second plasmid and thereby curing the host of the second plasmid, and repeating as necessary to complete the desired genomic edits. In some cases, the first and/or second plasmid comprise at least one donor poly nucleotide (e.g., wherein the donor polynucleotides of the first and second plasmid are different). As conclusively demonstrated herein for the first time, this sequential genomic editing approach dramatically improves gene editing efficiency in the Corynebacterium host.

In another aspect, the disclosure provides methods of improving CRLSPRICas9 editing in gram positive bacteria, such as by placing the guide RNA used in CRISPR/Cas9 editing under the control of a promoter disclosed herein.

In another aspect, the disclosure provides a Corynebacterium host comprising: a) a first plasmid comprising a promoter operably linked to a first guide RNA, and b) a first donor polynucleotide having a right homology arm sequence and a left homology arm sequence, wherein each homology ann sequence is homologous to a target sequence in a Counebacterium genome, wherein said host expresses said first guide RNA in conjunction with a Cas9 polypeptide. In some cases, the Corynebacterium host is a Corynebacterium ghtiamicum host. In some cases, the Corynebacterium plutonic:um host is Corynebacterium giutamicum strain NRRL-B11474.

In some embodiments, the first plasmid comprises a replication origin selected from the group consisting of a pCASE1 replication origin and a pCG1 replication origin. In some embodiments, the first plasmid comprises a pCASEI replication origin. In some embodiments, said first donor polynucleotide is provided on said first plasmid. In some embodiments, said first donor polynucleotide is provided on a second plasmid. In some embodiments, said first donor polynucleotide is provided as a linear nucleic acid fragment. In some embodiments, said first plasmid further comprises one or more additional guide RNAs operably linked to said first promoter or one or more additional guide RNAs operably linked to one or more additional promoters.

In some embodiments, a first donor polynucleotide is provided on the first plasmid and said first plasmid further comprises one or more additional donor polynucleotides. In some embodiments, said first donor polynucleotide is provided on the first plasmid and one or more additional donor polynucleotides are provided on a second plasmid. In some embodiments, each of the first, second, third, etc. donor polynucleotides comprises at least one mutation sequence which comprises a mutation selected from the group consisting of: a single nucleotide insertion; an insertion of two or more nucleotides; an insertion of a nucleic acid sequence encoding one or more proteins; a single nucleotide deletion; a deletion of two or more nucleotides; a deletion of one or more coding sequences; a substitution of a single nucleotide; a substitution of two or more nucleotides; and any combination thereof.

In some embodiments, said at least one mutation sequence comprises a mutation of a Cas9 PAM or seed region. In some cases, the at least one mutation sequence comprises a mutation of a Cas9 PAM. In some cases, at least one mutation sequence comprises a mutation of a Cas9 seed region. In some cases, at least one mutation sequence comprises a mutation of a Cas9 seed region and at least one mutation of a sequence comprises a mutation of a Cas9 PAM.

In some embodiments, said Cas9 polypeptide is expressed from a Cas9 polypeptide encoding sequence operably linked to a promoter. In some cases, the Cas9 promoter is constitutive. In some embodiments, the constitutive Cas9 promoter is selected from a group consisting of Pcg2613 or Pcg0007 or Pcg0047 or Pcg1133 or PTrc. In some cases, the Cas9 promoter is inducible. In some embodiments, the inducible Cas9 promoter is selected from a group consisting of PTet1 or PTet3 or PLac1 or PLac2 or PAra1. In some cases, the Cas9 promoter is differentially inducible as compared to an inducible promoter operably linked to a guide RNA and/or an inducible promoter operably linked to a donor polynucleotide. In some embodiments, said Cas9 polypeptide encoding sequence is in a plasmid. In some embodiments, said first plasmid further comprises said Cas9 polypeptide encoding sequence operably linked to a promoter. In some embodiments, said second plasmid further comprises said Cas9 polypeptide encoding sequence operably linked to a promoter. In some embodiments, said plasmid comprising said Cas9 polypeptide encoding sequence comprises a counterselection marker. In some cases, said counterselection marker can be differentially counterselected in comparison to another counterselection marker on a different (e.g., first or second) plasmid. In some embodiments, said Cas9 polypeptide encoding sequence operably linked to a promoter is integrated into the genome of the Comiebacterium host. In some cases, said Cas9 polypeptide encoding sequence comprises a coding sequence optimized for expression in a Corynebacterium species.

In some embodiments, said right and left homology arm sequences are at least 25 base pairs, preferably at least 150, 200, 250, or 300 base pairs, more preferably at least 350, 400, 450, 500, 550, or 600 base pairs. In some embodiments, said first, second, and/or additional promoters are selected from the group consisting of: Pcg2613 or Pcg0007 or Pcg0047 or Pcg1133 or PTet1 or PTet3 or PLac1 or PLac2 or PAra1 or PTrc. In some embodiments, said first, second, and/or additional promoters are selected from the group consisting of an endogenous Corynbacterium promoter, a promoter that is heterologous to the Corynebacterium host, a synthetic promoter, an inducible promoter, and a constitutive promoter. In some embodiments, said first and/or second promoter is Pcg2613. In some embodiments, said first promoter is Pcg2613. In some embodiments, the guide RNA is a single-molecule guide IRNA (sgRNA). In some embodiments, the guide RNA is a dual-molecule guide RNA, e.g., crRNA and tracrRNA. In some embodiments, said first guide RNA comprises a CRISPR RNA (crRNA) and a trans-activating RNA (tracrRNA.) and a first spacer sequence of at least 20 nucleotides wherein said first spacer sequence is at least 80%, 85%, 90%. 95%, or 100% complementary to said Corynebacterium target sequence. In some embodiments said first guide RNA comprises a sgRNAsgRNA. In some embodiments, said first guide RNA comprises a CRISPR RNA (crRNA) and a trans-activating crRNA (tracrRNA). In some embodiments, said first guide RNA comprises a single gRNA (sgRNA).

In some embodiments, the host comprises at least two different inducible promoters operably linked to at least two different guide RNA sequences. In some embodiments, the first plasmid comprises a counterselection marker.

In some embodiments, the invention provides a Corynebacterium host comprising: a first plasmid comprising a promoter operably linked to a first guide RNA; and a first donor polynucleotide having a right homology arm sequence and a left homology arm sequence, wherein each homology arm sequence is homologous to a target sequence in a Corynebacterium genome; wherein said host expresses said first guide RNA in conjunction with an RNA-guided DNA endonuclease polypeptide. In other embodiments, the invention provides a Corynebacterium host comprising: a first plasmid comprising a promoter operably linked to a sequence for expressing an RNA-guided DNA endonuclease polypeptide; and a first guide RNA; wherein said host expresses said RNA-guided DNA endonuclease polypeptide in conjunction with a first donor polynucleotide having a left homology arm sequence and a right homology arm sequence each homologous to a Corynebacterium target sequence in said host, said first donor polynucleotide including at least one mutation sequence flanked by said left and right homology arm sequences. In some embodiments, the Corynebacterium host is a Corynebacterium count host. In some embodiments, the Corynebacterium glutamicum host is Corynebacterium glutamieum strain NRRL-B11474. In some embodiments, the RNA-guided DNA endonuclease is selected from the group consisting of Cas9, Cas12a, Cas12b, Cas12c, Cas12d, Cas12e, Cas12h, Cas13a, Cas13b, Cas13c, Cpf1, and MAD7, or homologs, orthologs, or paralogs thereof. In some embodiments, the RNA-guided DNA endonuclease is Cas9. In some embodiments, the RNA-guided DNA endonuclease is provided by plasmid-based presentation. In some embodiments, the RNA-guided DNA endonuclease is integrated into the genome of said Corynebacterium species. In some embodiments, the first plasmid comprises a replication origin selected from the group consisting of a pCASEI replication origin and a pCGI replication origin. In some embodiments, the first plasmid comprises a pCASEI replication origin. In some embodiments, the first donor polynucleotide is provided on said first plasmid. In some embodiments, the first donor polynucleotide is provided on a second plasmid. In some embodiments, the first donor polynucleotide is provided as a linear nucleic acid fragment. In some embodiments, the first plasmid further encodes the first guide RNA or one or more additional guide RNAs operably linked. to said first promoter, or one or more additional guide RNAs operably linked to one or more additional promoters. In some embodiments, the first donor polynucleotide is provided on said first plasmid and wherein said first plasmid further comprises one or more additional donor polynucleotides. In some embodiments, the first donor polynucleotide is provided on a second plasmid and wherein said second plasmid further comprises one or more additional donor polynucleotides.

In some embodiments, the invention provides a Corynebacterium host as described herein, wherein the first donor polynucleotide comprises at least one mutation sequence which comprises a mutation selected from the group consisting of: a single nucleotide insertion; an insertion of two or more nucleotides; an insertion of a nucleic acid sequence encoding one or more proteins; a single nucleotide deletion; a deletion of two or more nucleotides; a deletion of one or more coding sequences; a substitution of a single nucleotide; a substitution of two or more nucleotides; two or more non-contiguous insertions, deletions, and/or substitutions; and any combination thereof. In some embodiments, the host comprises a second donor polynucleotide, and wherein the second donor polynucleotide comprises at least one mutation sequence which comprises a mutation selected from the group consisting of: a single nucleotide insertion; an insertion of two or more nucleotides; an insertion of a nucleic acid sequence encoding one or more proteins; a single nucleotide deletion; a deletion of two or more nucleotides; a deletion of one or more coding sequences; a. substitution of a single nucleotide; a substitution of two or more nucleotides; two or more non-contiguous insertions, deletions, and/or substitutions; and any combination thereof. In sonic embodiments, the at least one mutation sequence comprises a mutation of an RNA-guided DNA endonuclease protospacer-adjacent motif (PAM) or seed region. In some embodiments, the RNA-guided DNA endonuclease polypeptide is expressed from a RNA-guided DNA endonuclease polypeptide encoding sequence operably linked to a constitutive or an inducible promoter. In some embodiments, the first plasmid further comprises said RNA-guided DNA endonuclease polypeptide encoding sequence operably linked to a constitutive or an inducible promoter. In some embodiments, the RNA-guided DNA endonuclease polypeptide encoding sequence comprises a coding sequence optimized for expression in a Corynebacterium species. In some embodiments, the right and left homology arm sequences are at least 25 base pairs, preferably at least 150, 200, 250, or 300 base pairs, more preferably at least 350, 400, 450, 500, 550, or 600 base pairs. In some embodiments, the promoter is selected from the group consisting of an endogenous Corynebacterium promoter, a promoter that is heterologous to the Corynebacterium host, a synthetic promoter, an inducible promoter, and a constitutive promoter. In some embodiments, the promoter is Pcg2613. In some embodiments, the first guide RNA comprises a. CRISPR RNA (crRNA) and a trans-activating crRNA (tracrRNA). In some embodiments, the first guide RNA comprises a single gRNA (sgRNA). In some embodiments, the first guide RNA comprises a CRISPR RNA (crRNA) and a trans-activating RNA (tracrRNA) and a first spacer sequence of at least 20 nucleotides wherein said first spacer sequence is at least 80%, 85%, 90%, 95%, or 100% complementary to said Corynebacterium target sequence. in some embodiments, the host comprises at least two different inducible promoters operably linked to at least two different guide RNA sequences. In some embodiments, the first plasmid comprises a counterselection marker. In some embodiments, the host comprises a set of proteins from one or more heterologous recombination systems in said host. In some embodiments, the host comprises a set of proteins from a lambda red recombination system, a Rec ET recombination system, any homologs, orthologs or paralogs of proteins from a lambda red recombination system or a Rec ET recombination system, or any combination thereof.

### BRIEF DESCRIPTION OF THE DRAWINGS

FIG. 1 presents photographs of plates of NRRL-1311474 C. glutamicum that have been transformed with plasmids containing a sg,RNA targeting three loci in the C. glutamicum genome (rpsL, c.g303 1 and cg3404). The top panels depict results with control strains of NRRL-B11474 C. glutamicum. The bottom panels depict Cas9-containing NRRL-B11474 C. glutamicum that has been transformed with a guide plastid d identical to the guide plasmid in the control strain above. Lethality of the Cas9 and sgRNA complex is apparent from a vast decrease in the number of transformants where the sgRNA is targeting a sequence that is present in the genome, thus showing CRISPR/Cas9 is active and the sgRNA sequences are functional.

FIG. 2 shows colony counts resulting from sgRNA constructs that have been transformed into strains containing or lacking the Cas9 gene as indicated. Plasmids encoding a sgRNA targeting one of five loci cg0167, cg3031, (4′3404, gdh4, or rpsL) were constructed using two different C. Wuiamicum origins of replication (pCASE1, pCG-1). Plasmids did not contain donor fragments. Competent NRRL-B11474 C. glutamicum cells containing integrated Cas9 (Cas9 NRRL-B11474), or wild type control (WT NRRL-B11474) were transformed with the sgRNA plasmids, and serial dilutions were plated on selective media. The indicated colony counts are an average of the colony counts for two independent transformations. Significantly lower colony counts were observed in Cas9-containing strains as compared to the WT control strain, indicating that the sgRNAs and Cas9 protein is functional for all loci tested.

FIG. 3 is a schematic of exemplary sgRNA and donor configurations that may be used to introduce SNPs in NRRL-B11474 C. glutamicum with integrated. Cas9. A. In one embodiment, a plasmid containing a C. glutamicum origin of replication, a sgRNA, a resistance marker, and a donor fragment with left (L) and right (R) homology arms that flank a target SNP may be used. B. In another embodiment, a plasmid containing a C. glutamicum origin of replication, a sgRNA, a resistance marker, and a separate PCR product containing a donor fragment with left (L) and right (R) homology arms that flank a target SNP may be used.

FIG. 4 is a schematic of exemplary sgRNA and donor configurations that may be used to create knockouts in C. glutamicum with integrated Cas9. A. In one embodiment, a plasmid containing a C. glutamicum origin of replication, a sgRNA, a resistance marker, and a donor fragment with left (L) and right (R) homology arms may be used. Be In another embodiment, a. plasmid containing a C. glutamicum origin of replication, a sgRNA, and a resistance marker; and, a separate plasmid without a C. glutamicum origin of replication that contains a donor fragment with left (L) and right (R) homology arms may be used. C. In another embodiment, a plasmid containing a C. glutamicum origin of replication, a sgRNA, and resistance marker with a separate PCR product containing a donor fragment with left (L) and right (R) homology arms.

FIG. S is a schematic of an exemplary sgRNA and donor configurations that may be used to create insertions in C. glutamicum with integrated Cas9. A. A plasmid containing a C. glutamicum origin of replication, a sgRNA, resistance marker, and a donor fragment with left (L) and right (R) homology arms that flank an insert.

FIG. 6 depicts coverage plots showing a colony with three successfully integrated SNPs at the rpsL locus, and an unedited wild type colony. Competent C. glutamicum cells were transformed with a plasmid encoding a sgRNA targeting the ipsl, locus and a donor fragment encoding three separate SNPs. 1. a SNP mutating the PAM region and preventing further recognition by the sgRNA/Cas9 complex. 2. a SNP mutating the seed region of the protospacer recognition sequence, 10 by from the mutation in the PAM region. 3. a SNP in the ipsL, open reading frame, 65 by from the PAM site. Primers hybridizing outside the homology arms on the donor fragment were used to amplify the rpsL region from screened colonies. A tagmentation library was generated with this amplicon, and submitted for high-throughput sequencing. A. Sequencing reads from a colony that successfully incorporated all three SNPs, aligned to the rpsL donor fragment without errors. B. Sequencing reads from an unedited WT colony, which align to the rpsL donor fragment and exhibit sequence discrepancy at each of the engineered SNP sites. C. A schematic of an exemplary rpsL donor fragment encoding three separate SNPs is shown.

FIG. 7 illustrates results of RNA-guided endonuclease editing in C. glutamicum according to an embodiment of the invention. NRRL-B11474 C. glutamicum cells containing an integrated Cas9 gene were transformed with a plasmid containing the pCASE1 origin. The pCASEI plasmid encoded a sgRNA that targets one of three loci, and a matching donor fragment encoding a SNP that scrambles the PAM locus, the SNP flanked on either side with 125 bp of homologous sequence. Colonies were screened by PCR amplification, tagmentation, and next generation sequencing, and percent edited was calculated by tabulating total number of sequence-confirmed edited colonies over total colonies screened.

FIG. 8 illustrates results of RNA-guided endonuclease editing in C. glutamicum according to an embodiment of the invention. Plasmids containing either the pCASE1 or pCG1 origin of replication were constructed to encode a sgRNA that targets one of three loci (cg0167, cg3404, rpsL), and a donor fragment designed to introduce an SNP at the targeted locus. Donor fragments were constructed with a range of homology arm lengths flanking either side of the intended SNP, from 25 bp up to 125 bp. Colonies were screened by PCR amplification, tagmentation, and next generation sequencing and percent edited calculated by tabulating total number of sequence-confirmed edited colonies over total colonies screened.

FIG. 9 illustrates results of RNA-guided endonuclease genome editing in C. glutamicum according to an embodiment of the invention. Plasmids containing either the pCASE1 or pCGI origin of replication were constructed to encode a sgRNA that targets one of three loci (eg30.31, cg3404, gdhA) and a donor fragment designed to introduce a small insertion of 100 bp at the target locus. Donor fragments were constructed with a range of homology arm lengths flanking either side of the intended insertion, from 25 bp up to 2000 bp. Colonies were screened by PCR amplification, tagmentation, and next generation sequencing, and percent edited was calculated by tabulating total number of sequence-confirmed edited colonies over total colonies screened.

FIG. 10 is a schematic representation of an exemplary configuration to generate edits in a C. glutamicum genome with an RNA-guided endonuclease. This configuration uses a dsDNA donor fragment and a replicating helper pla.smid expressing RecET with a separate replicating plasmid that encodes sgRNA.

FIG. 11 shows colony screening results of RNA-guided endonuclease editing by delivery of a PCR-amplified donor fragmentand sgRNA encoded in replicating plasmid. Colonies were screened using PCR and verified via Sanger sequencing analysis. Edited colonies were knockouts that remove 702 by from the cg303.1 locus. Each bar represents the mean percentage of colonies that screened positive for the intended edit, and points represent percent colonies edited from individual transformation events.

FIG. 12 depicts data demonstrating the effectiveness of two different C. glutamicum origins of replication for making SNPs and small inserts by CRISPR/Cas9 editing with a plasmid-based system. Two sets of editing plasmids were constructed to introduce either a SNP (A. & C.) or a 100 bp insert (B. & at one of three loci. Plasmids contain either the pCASE1 or pCG1 origin of replication. All plasmids also contain a sgRNA specific to the target locus, and a donor fragment that contains either 125 by of homology on either side of the SNP, or 500 bp on either side of the insertion. Plasmids were transformed into a NRR1,1311474 C. glutamicum strain carrying an integrated, constitutively expressed copy of the Cas9 gene, and up to 8 colonies were picked for screening by next generation sequencing (NGS). Two biological replicates were averaged for each editing construct. A. Percentage of colonies screening positive for intended SNP introduction, by target locus and C. glutamicum origin of replication. B. Percentage of colonies screening positive for intended 100 by insertion, by target locus and C. glutamicum origin of replication. C. D. Comparison of means by student's t test of small insertion editing by C. glutamicum origin of replication, demonstrating a statistically significant increase in insertion editing efficiency when using the pCASE1 origin.

FIG. 13 illustrates deletion of 702 bp from C. glutamicum genomic locus cg3031 and results from an evaluation of guide RNA design parameters. In this experiment plasmids containing a donor fragment designed to introduce a 702 by deletion in the cg3031 locus and a sgRNA targeting the deletion region were introduced into a Cas9 expressing NRRL-B11474 C. glutamicum strain. Following transformation of the plasmids into the Cas9 expressing strain, colonies were screened for the presence of deletion of 702 bp deletion from the cg303/ locus using PCR and DNA-fragment analysis. The wild-type cg3031 locus produces a 1648 by band while colonies possessing the target deletion in the cg3031 locus produce a 946 bp band. PCR fragment analysis data shows the deletion of 702 bp from the cg3031 locus in 6 out of 8 colonies.

FIG. 14 is a graph comparing transformation efficiency for five C. glutamicum origins of replication. Center lines of diamonds represent population mean and outer diamond lines are 95% confidence intervals. Unexpectedly, plasmid systems containing CASE1 and CG1 replication origins exhibited statistically significant higher (P<0.05 via Tukey-Kramer) transformation efficiency as compared to pBL1, pCC1, and pNG2 plasmids.

FIG. 15 illustrates coverage plots demonstrating multiplexed editing at two loci, cg3404 and rpsL. The plots depict number of reads on the y axis and chromosomal coordinate on the x axis. Vertical markers (lighter shade) indicate disagreements between the aligned reads and reference sequence. When reads from a mutant colony are mapped to the wild-type reference sequence there are two disagreements at the cg3404 locus and three disagreements at the rpsL locus, indicative of editing at these loci. There are no disagreements at the cg0167 locus—a locus that was not targeted for editing. Conversely, when reads from a mutant colony are mapped to cg0167, cg3404, and rpsL, the reads align perfectly at cg3404 and rpsL (as expected) and disagree with mutations at cg0167 (as expected).

### DETAILED DESCRIPTION OF THE INVENTION

### Definitions

While the following terms are believed to be well understood by one of ordinary skill in the art, the following definitions are set forth to facilitate explanation of the presently disclosed subject matter.

The term “a” or “an” refers to one or more of that entity, i.e., can refer to a plural referents. As such, the terms “a” or “an”, “one or more” and “at least one” are used interchangeably- herein. In addition, reference to “an element” by the indefinite article “a” or “an” does not exclude the possibility that more than one of the elements is present, unless the context clearly requires that there is one and only one of the elements.

Unless otherwise indicated, the term “about” refers to a variation in the indicated parameter of +10%,

The terms “genetically modified host cell,” “recombinant host cell,” and “recombinant strain” are used interchangeably herein and refer to host cells that have been genetically modified by the CRISPR-mediated methods of the present disclosure. Thus, the terms include a host Corynebacterium cell that has been genetically altered, modified, or engineered, such that it exhibits an altered, modified, or different genotype and/or phenotype (e.g., when the genetic modification affects coding nucleic acid sequences of the microorganism), as compared to the naturally-occurring microorganism from which it was derived. It is understood that the terms refer not only to the particular recombinant microorganism in question, but also to the progeny or potential progeny of such a microorganism.

The term “genetically engineered” may refer to any manipulation of a host Corynebacterium cell's genome (e.g., by insertion, deletion or substitution of nucleic acids).

The terms “polynucleotide” and “nucleic acid” are used interchangeably herein and refer to a polymeric form of nucleotides of any length, either ribonucleotides or deoxyribonucleotides, or analogs thereof. These terms refer to the primary structure of the molecule, and thus include double- and single-stranded DNA, as well as double- and single-stranded RNA. They also include modified nucleic acids such as methylated and/or capped nucleic acids, nucleic acids containing modified bases, backbone modifications, and the like.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homolog” or “ortholog” is known in the art and refers to related sequences that share a common ancestor or family member and are determined based on the degree of sequence identity. The terms “substantially similar” and “corresponding substantially” are used interchangeably herein. They refer to nucleic acid fragments wherein differences in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid. fragment relative to the initial, unmodified fragment. It is therefore understood, as those skilled in the art will appreciate, that the disclosure encompasses more than the specific exemplary sequences. These terms “homologous” or “homolog” or “ortholog” or “substantially similar” or “corresponding substantially” can describe the relationship between a gene found in one species, subspecies, variety, cultivar or strain and the corresponding or equivalent gene in another species, subspecies, variety, cultivar or strain.

For purposes of this disclosure homologous sequences are compared. “Homologous sequences” or “homologs” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as NCBI BLAST (Basic Local Alignment Search Tool), using default parameters.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to he used will depend on the particular application. A portion of a nucleic acid useful as a hybridization probe or targeting region of a guide RNA may be as short as 12 nucleotides; in some aspects, it is or is about 15, 20, or 25 nucleotides. A portion of a polypeptide useful as an epitope may be as short as 4 amino acids. A portion of a polypeptide that performs the function of the full-length polypeptide would generally be longer than 4 amino acids. In some cases, a portion of a polypeptide that performs the function of the full-length polypeptide contains 1, 2, 3, 4, 5, 6, 7, 8, 9, or 10 amino acids deleted from the N and/or C-terminus.

As used herein, “promoter” refers to a DNA sequence capable of controlling the expression of a coding sequence or functional RNA. The promoter sequence may consist of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Non-limiting promoter sequences suitable for use in the methods of the present specification are provided below at Table 1: Exemplary Promoters to drive guide RNA expression.

TABLE 1
Exemplary Promoters to drive guide RNA or RNA-
guided endonuclease (e.g., Cas9) expression

| | | SEQ |
|---|---|---|
| | | ID |
| Promoter | SEQUENCE | NO: |
| Pcg2613 | CGTCAAGATCACCCAAAACTGGTGG | 4 |
| | CTGTTCTCTTTTAAGCGGGATAGCA | |
| | TGGGTTCTT | |
| Pcg0007 | TGCCGTTTCTCGCGTTGTGTGTGGT | 5 |
| | ACTACGTGGGGACCTAAGCGTGTAA | |
| | GATGGAAACGTCTGTATCGGATAAG | |
| | TAGCGAGGAGTGTTCGTTAAAA | |
| Pcg0047 | TAACTACATTGAGCGAAATGCCAAC | 6 |
| | CACATGTCCCATGCTTTTACTAATG | |
| | TGGGGTCTTAGAAGAAAGCGACCAA | |
| | TTTAAGGAGAGTTGAAT | |
| Pcg1133 | AGTGAACCCATACTTTTATATATGG | 7 |
| | GTATCGGCGGTCTATGCTTGTGGG | |
| PTet1 | TCCCTATCAGTGATAGAGATTGACA | 8 |
| | TCCCTATCAGTGATAGAGATACTGA | |
| | GCACATCAGCAGGACGCACTGACC | |
| PTet3 | TCGTCAAGATCACCCAAAACTGGTG | 9 |
| | GCTGTTCTCTTTTAAGCGGGATAGC | |
| | ATGGGTTCTTATCCCTATCAGTGAT | |
| | AGAGA | |
| PLac1 | TTGACAATTAATCATCGGCTCGTAT | 10 |
| | AATGTGTGGAATTGTGAGCGGATAA | |
| | CAATTTCACACA | |
| PLac2 | CTCGAGGGTAAATGTGAGCACTCAC | 11 |
| | AATTCATTTTGCAAAAGTTGTTGAC | |
| | TTTATCTACAAGGTGTGGCATAATG | |
| | TGTGTAATTGTGAGCGGATAACAAT | |
| | T | |
| PAra1 | ACTTTTCATACTCCCGCCATTCAGA | 12 |
| | GAAGAAACCAATTGTCCATATTGCA | |
| | TCAGACATTGCCGTCACTGCGTCTT | |
| | TTACTGGCTCTTCTCGCTAACCAAA | |
| | CCGGTAACCCCGCTTATTAAAAGCA | |
| | TTCTGTAACAAAGCGGGACCAAAGC | |
| | CATGACAAAAACGCGTAACAAAAGT | |
| | GTCTATAATCACGGCAGAAAAGTCC | |
| | ACATTGATTATTTGCACGGCGTCAC | |
| | ACTTTGCTATGCCATAGCATTTTTA | |
| | TCCATAAGATTAGCGGATCCTACCT | |
| | GACGCTTTTTATCGCAACTCTCTAC | |
| | TGTTTCTCCATACCCGTTTTTTTGG | |
| | GAATTCGAGCTCTAAGGAGGTTATA | |
| | AAAA | |
| PTrc | GAGCTGTTGACAATTAATCATCCGG | 13 |
| | CTCGTATAATGTGTGGAATTGTGAG | |
| | CGGATAACAATTTCACACAGGAAAC | |
| | AGCGCCGCTGAGAAAAAGCGAAGCG | |
| | GCACTGCTCTTTAACAATTTATCAG | |
| | ACAATCTGTGTGGGCACTCGACCGG | |
| | AATTATCGATTAACTTTATTATTAA | |
| | AAATTAAAGAGGTATATATTAATGT | |
| | ATCGATTAAATAAGGAGGAATAAAC | |
| | C | |

As used herein, the terms “endogenous,” and “native” refer to the naturally occurring copy of a gene or promoter.

As used herein, the term “naturally occurring” refers to a gene derived from a naturally occurring source. In some aspects a naturally occurring gene refers to a gene of a wild type (non-transgene) gene, whether located in its endogenous setting within the source organism, or if placed in a “heterologous” setting, when introduced in a different Organism. Thus, for the purposes of this disclosure, a “non-naturally occurring” gene is a gene that has been mutated or otherwise modified, or synthesized, to have a different sequence from known natural genes, in some aspects, the modification may be at the protein level (e.g., amino acid substitutions). In other aspects, the modification may be at the DNA level, without any effect on protein sequence (e.g., codon optimization).

As used herein, the term “heterologous” refers to an amino acid or a nucleic acid sequence (e.g., gene or promoter), which is not naturally found in the particular organism or is not naturally found in a particular context (e.g., genomic or plasmid location) in the particular organism. For example, a native promoter or other nucleic acid sequence of C. glutamicum can be heterologous when operably linked to a nucleic acid sequence it is not operably linked to in a wild-type C. glutamicum, or when it is delivered in a non-native form such as in a heterologous plasmid or a heterologous nucleic acid fragment.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system. Artificially mutated variants of endogenous genes are considered “exogenous” for the purposes of this disclosure.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used.

The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern blot analysis of DNA, Northern blot analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an traNA or a protein (precursor or mature).

The term “operably linked” means in this context the sequential arrangement of the promoter polynucleotide according to the disclosure with a further oligo- or polynucleoti de, resulting in transcription of said further polynucleotide. In some aspects, the promoter sequences of the present disclosure are inserted just prior to a gene's 5′UTR, or open reading frame. In other aspects, the operably linked promoter sequences and gene sequences of the present disclosure are separated by one or more linker nucleotides.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells,a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “target nucleic acid” as used herein is a polynucleotide (e.g., RNA, DNA) that includes a target site or “target sequence.” The terms “target site” or “target sequence” are used interchangeably herein to refer to a nucleic acid sequence present in a target nucleic acid to which a targeting segment of a subject guide nucleic acid will bind, provided sufficient conditions for binding exist. Suitable hybridization conditions include physiological conditions normally present in a cell. For a double stranded target nucleic acid, the strand of the target nucleic acid that is complementary to and hybridizes with the guide nucleic acid is referred to as the “complementary strand”; while the strand of the target nucleic acid that is complementary to the “complementary strand” (and is therefore not complementary to the guide nucleic acid) is referred to as the “noncomplementary strand” or “non-complementary strand”. In embodiments where the target nucleic acid is a single stranded target nucleic acid (e.g., single stranded DNA (ssDNA), single stranded RNA (ssRNA)), the guide nucleic acid is complementary to and hybridizes with single stranded target nucleic acid.

A nucleic acid molecule that binds to an RNA-guided endonuclease (e.g., the Cas9 Polypepti de) and targets the polypeptide to a specific location within the target nucleic acid is referred to herein as a “guide nucleic acid”. When the guide nucleic acid is an RNA molecule, it can be referred to as a “guide RNA” or a “gRNA”. A guide nucleic acid comprises two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. For example, in some embodiments the protein-binding segment (described below) of a guide nucleic acid is one nucleic acid molecule (e.g., one RNA molecule) and the protein-binding segment therefore comprises a region of that one molecule. In other embodiments, the protein-binding segment (described below) of a guide nucleic acid comprises two separate molecules that are hybridized along a region of complementarity. As an illustrative, non-limiting example, a protein-binding segment of a guide nucleic acid that comprises two separate molecules can comprise (i) base pairs 40-75 of a first molecule (e.g., RNA molecule, DNA/RNA hybrid molecule) that is 100 base pairs in length; and (ii) base pairs 10-25 of a second molecule (e.g., RNA molecule) that is 50 base pairs in length. The definition of “segment,” unless otherwise specifically defined in a particular context, is not limited to a specific number of total base pairs, is not limited to any particular number of base pairs from a given nucleic acid molecule, is not limited to a particular number of separate molecules within a complex, and may include regions of nucleic acid molecules that are of any total length and may or may not include regions with complementarity to other molecules.

The first segment (targeting segment) of a guide nucleic acid (e.g., guide RNA or gRNA) comprises a nucleotide sequence that is complementary to a specific sequence (a target site) within a target nucleic acid (e.g., a target ssRNA, a target ssDNA., the complementary strand of a double stranded target DNA, etc.). The protein-binding segment (or “protein-binding sequence”) interacts with an RNA-guided endonuclease (e.g., Cas9) polypeptide. Site-specific binding and/or cleavage of the target nucleic acid can occur at locations determined by base-pairing complementarity between the guide nucleic acid (e.g., guide RNA) and the target nucleic acid.

The protein-binding segment of a subject guide nucleic acid comprises two complementary stretches of nucleotides that hybridize to one another to form a double stranded RNA duplex (dsRNA duplex).

A subject guide nucleic acid guide RNA)) linked to a donor polynucleotide forms a complex with a subject RNA-guided endonuclease (e.g., Cas9) (i.e., binds via non-covalent interactions). The guide nucleic acid (e.g., guide RNA) provides target specificity to the complex by comprising a nucleotide sequence that is complementary to a sequence of a target nucleic acid. Thus, the RNA-guided endonuclease (e.g., Cas9) of the complex provides site-specific or “targeted” activity by virtue of its association with the protein-binding segment of the guide nucleic acid.

In some embodiments, a subject guide nucleic acid (e.g., guide RNA) comprises two separate nucleic acid molecules and is referred to herein as a “dual guide nucleic acid.” In some embodiments, the subject guide nucleic acid is a single nucleic acid molecule (single polynucleotide) and is referred to herein as a “single guide nucleic acid.” The term “guide nucleic acid” is inclusive, referring to both dual guide nucleic acids and to single guide nucleic acids and. the term “guide RNA” is also inclusive, referring to both dual guide RNA (dgRNA) and single guide RNA (sgRNA).

In some embodiments, a guide nucleic acid is a DNA/RNA hybrid molecule. In such embodiments, the protein-binding segment of the guide nucleic acid is RNA and forms an RNA duplex. However, the targeting segment of a guide nucleic acid can be DNA. Thus, if a DNA/RNA hybrid guide nucleic acid is a dual guide nucleic acid, the targeting segment can be DNA and the duplex-forming segment can be RNA. In such embodiments, the duplex-forming segment of the “activator” molecule can be RNA (e.g., in order to form an RNA-duplex with the duplex-forming segment of the targeting segment), while nucleotides of the “activator” molecule that are outside of the duplex-forming segment can be DNA (in which case the activator molecule is a hybrid DNA/RNA molecule) or can be RNA (in which case the activator molecule is RNA). If a. DNA/RNA hybrid guide nucleic acid is a single guide nucleic acid, then the targeting segment can be DNA, the duplex-forming segments (which make up the protein-binding segment) can be RNA, and nucleotides outside of the targeting and duplex-forming segments can be RNA or DNA.

An exemplary dual guide nucleic acid comprises a CRISPR-RNA (crRNA) molecule and a corresponding trans-activating crRNA (tracrRNA) molecule. The crRNA molecule comprises both the targeting segment (single stranded) of the guide nucleic acid and a stretch (“duplex-forming segment”) of nucleotides that forms one half of the dsRNA duplex of the protein-binding segment of the guide nucleic acid. The corresponding tracrRNA molecule comprises a stretch of nucleotides (duplex-forming segment) that forms the other half of the dsRNA duplex of the protein-binding segment of the guide nucleic acid. In other words, a stretch of nucleotides of a crRNA molecule are complementary to and hybridize with a stretch of nucleotides of a tracrRNA molecule to form the dsRNA duplex of the protein-binding domain of the guide nucleic acid. The crRNA-like molecule additionally provides the single stranded targeting segment. Thus, the crRNA and the tracrRNA (as a corresponding pair) hybridize to form a dual guide nucleic acid. The exact sequence of a given crRNA or tracrRNA molecule is characteristic of the species in which the RNA molecules are found.

The term “protospacer” refers to the DNA sequence targeted by a crRNA guide strand. In some aspects the protospa.cer sequence hybridizes with the crRNA guide sequence of a CRISPR complex.

The “protospa.cer-adjacent motif” or “PAM” sequence is a 2-6 base pair DNA sequence immediately following the DNA sequence targeted by an RNA-guided endonucl ease (e.g., Cas9). The PAM sequences is required for cleavage of the target nucleic acid and varies depending on the source of the RNA-guided endonuclease (e.g., Cas9). For example, in case of the Streptococcus pyogenes Cas9 the PAM sequence is NGG. In aspects of the present disclosure, the PAM sequences is mutated by the donor polynucleotide such that further cleavage of the target site is prevented.

In some instances, a component, e.g., a nucleic acid component (e.g.,, a guide nucleic acid, etc.); a protein component (e.g., an RNA-guided endonuclease, a Cas9 polypeptide, a variant RNA-guided endonuclease, a variant Cas9 polypeptide); and the like) includes a label moiety. The terms “label”, “detectable label”, or “label moiety” as used herein refer to any moiety that provides for signal detection and may vary widely depending on the particular nature of the assay. Label moieties of interest include both directly detectable labels (e.g., a fluorescent label) and indirectly detectable labels (indirect labels, e.g., a binding pair member). A fluorescent label can be any fluorescent label, e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), inCherry, mTomato, inTangerine, and any fluorescent derivative thereof, etc.).

Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label) (e.g., 3H, 125I, 35S, 14C, or 32P); an enzyme (an indirect label) (e.g., peroxidase, alkaline phosphatase, galactosidase, luciferase, glucose oxidase, and the like); a fluorescent protein (a direct label) (e.g., green fluorescent protein, red fluorescent protein, yellow fluorescent protein, and any convenient derivatives thereof); a metal label (a direct label); a colorimetric label; a binding pair member; and the like. By “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Any given component, or combination of components can be unlabeled, or can be detectably labeled with a label moiety. In some embodiments, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bo'lag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Leficovits ed., Academic Press 1997); Cell and Tissue Culture: Laboratory Procedures in Biotechnology (Doyle & Griffiths, John Wiley & Sons 1998); and Current Protocols in Molecular Biolgoy (Ausubel et al. eds., John Wiley & Sons 2003), including supplements 1-117, the disclosures of which are incorporated herein by reference.

### RNA-Guided Endonuelease Polypeptides

There are at least five main CRISPR system types (Type I, II, III, IV and V) and at least 16 distinct subtypes (Makarova, K.S., et al., Nat Rev Microbiol. 2015. Nat. Rev. Microbiol. 13, 722-736). CRISPR systems are also cla.ssified based on their effector proteins. Class 1 systems possess multi-subunit crRNA-effector complexes, whereas in class 2 systems all functions of the effector complex are carried out by a RNA-guided endonuclease (e.g., Cas9). As described in the Examples, the present disclosure advantageously employs Type II CRISPR RNA-guided endonucleases, such as Cas9 polypeptides, a variant thereof, and/or an ortholog. thereof. Persons having skill in the art will appreciate that aspects of the disclosure are applicable to other CRISPR/Cas systems besides those comprising Cas9 (e.g., Cpfl). Therefore,a suitable RNA-guided DNA endonuclease may be selected from, for example, Cas9, Cas12a, Cas12b, Cas12c, Cas12d, Cas12e, Cas12h, Cas13a, Cas13b, Cas13c, Cpf1, and MAD7, or homologs, orthologs, or paralogs thereof.

Suitable RNA-guided endonuclease polypeptides (e.g., Cas9 polypeptides) for use in the subject invention include naturally-occurring RNA-guided endonuclease polypeptides, e.g., Cas9 polypeptides (e.g., naturally occurs in bacterial and/or archaeal cells), or variant Cas9 polypeptides as discussed below. In one preferred embodiment, the Cas9 polypeptide is from Streptococcus pyogenes. In a particularly preferred embodiment, the RNA-guided endonuclease polypeptides (e.g., Cas9 polypeptide) has been codon optimized for Streptomyces as described in Cobb et al. ACS Synth. Biol. 4, 723-728 (2015).

As detailed herein, naturally occurring RNA-guided endonuclease polypeptides (e.g., Cas9 polypeptides) bind a guide nucleic acid, are thereby directed to a specific sequence within a target nucleic acid (a target site), and cleave the target nucleic acid (e.g., cleave dsDNA to generate a double strand break, cleave ssDNA, cleave ssRNA, etc.). A suitable RNA-guided endonuclease polypeptide (e.g., Cas9 polypeptide) will therefore comprise two portions, an RNA-binding portion and an activity portion. The RNA-binding portion interacts with a subject guide nucleic acid, and an activity portion exhibits site-directed enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc. In some embodiments the activity portion can exhibit reduced nuclease activity relative to the corresponding activity portion of a wild type RNA-guided endonuclease polypeptides (e.g., Cas9 polypeptide).

Assays to determine whether a protein has an. RNA-binding portion that interacts with a subject guide nucleic acid can be any convenient binding assay that tests for binding between a protein and a nucleic acid. Exemplary binding assays include binding assays (e.g., gel shift assays) that involve adding a guide nucleic acid and a RNA-guided endonuclease polypeptide (e.g., Cas9 polypeptide) to a target nucleic acid.

Assays to determine whether a protein has an activity portion (e.g., to determine if the polypeptide has nuclease activity that cleave a target nucleic acid) can be any convenient nucleic acid cleavage assay that tests for nucleic acid cleavage. Exemplary cleavage assays include, but are not limited to, adding a guide nucleic acid and a RNA-guided endonuclease polypeptide (e.g., Cas9 polypeptide) to a target nucleic acid and examining whether or not cleavage of the target nucleic acid has occurred via any suitable analytical technique, such as sequencing or PCR amplification.

RNA-guided endonuclease polypeptides Cas9 polypeptides) suitable for use in the present invention include variant RNA-guided endonuclease polypeptides Cas9 polypeptides). A variant RNA-guided endonuclease polypeptide (e.g., Cas9 polypeptide) has an amino acid sequence that differs by at least one amino acid (e.g., has a deletion, insertion, or substitution) when compared to the amino acid sequence of a wild type RNA-guided endonuclease polypeptide (e.g., Cas9 polypeptide), resulting in a modification of nuclease activity.

In some embodiments, the variant RNA-guided endonuclease polypeptide (e.g., Cas9 polypeptide) can cleave the complementary strand of a target nucleic acid but has reduced ability to cleave the non-complementary strand of a double stranded target nucleic acid. For example, the variant RNA-guided endonuclease polypeptide (e.g., Cas9 polypeptide) can have a mutation (amino acid substitution) that reduces the function of the RuvC domain. As a non-limiting example, in some embodiments, a variant Cas9 polypeptide has a DlOA mutation (e.g., aspartate to alanine at an amino acid position corresponding to position 10 of the Cas9 polypeptide encoded by the nucleic acid sequence of SEQ ID NO:3) and can therefore cleave the complementary strand of a double stranded target nucleic acid but has reduced ability to cleave the non-complementary strand of a double stranded target nucleic acid (thus resulting in a single strand break (SSB) instead of a double strand break (DSB) when the variant Cas9 polypeptide cleaves a double stranded target nucleic acid) (see, for example, Jinek et al., Science. 2012 Aug. 17; 337(6096):816-21).

In some embodiments, the variant RNA-guided endonuclease polypeptide (e.g., Cas9 polypeptide) can cleave the non-complementary strand of a double stranded target nucleic acid but has reduced ability to cleave the complementary strand of the target nucleic acid. For example, the variant RNA-guided endonuclease polypeptide (e.g., Cas9 polypeptide) can have a mutation (amino acid substitution) that reduces the function of the I-INI-i domain. As a non-limiting example, in some embodiments, the variant Cas9 polypeptide can have an H840A mutation (e.g., histidine to alanine at an amino acid position corresponding to position 840 of Streptococcus pyogenes and can therefore cleave the non-complementary strand of the target nucleic acid but has reduced. ability to cleave the complementary strand of the target nucleic acid (thus resulting in a SSB instead of a DSB when the variant Cas9 polypeptide cleaves a double stranded target nucleic acid).

In other embodiments, the RNA-guided endonuclease polypeptide Cas9 peptide) of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27;156(5):935-49; Jinek M. et al. Science. 2012 337:816-21; Jinek M. et al. Science. 2014 Mar 14;343(6176); and Chen et al. Nature. 2017 Oct. 19;550(7676):407-410; see also U.S. Pat. Pub. No. 2014/0068797; and 2016/0168592; see also PCT Pat. Pub. No. WO 2017/155717; WO 2017/147056; WO 2017/066175; WO 2017/040348; WO 2017/035416; WO 2017/015101; WO 2016/186953; and WO 2016/186745; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 9,840,713; 9,840,699; and 9,771,600. Each of the foregoing patents and publications are hereby incorporated by reference in the entirety for all purposes, which purposes include but are not limited to methods and compositions for targeting, cleaving, editing, modifying, or modulating expression of one or more nucleic acids with an RNA-guided nuclease, guide RNA, CRISPR associated protein, donor nucleic acid, and/or component of a CRISPR system.

Thus, in some embodiments, the systems and methods disclosed herein can be used with the wild type RNA-guided endonuclease polypeptides (e.g., Cas9 polypeptide) having double-stranded nuclease activity, RNA-guided endonuclease polypeptides (e.g., Cas9 variants) that act as single-stranded nickases, or other mutants with modified nuclease activity, As such, a RNA-guided endonuclease polypeptide (e.g., Cas9 polypeptide) that is suitable for use in the subject invention can be an enzymatically active RNA-guided endonuclease polypeptide (e.g., Cas9 polypeptide), e.g., can make single- or double-stranded breaks in a target nucleic acid, or alternatively can have reduced enzymatic activity compared to a wild-type RNA-guided endonuclease polypeptide (e.g., Cas9 polypeptide).

The RNA-guided endonuclease polypeptide (e.g, Cas9 polypeptide) can be provided to, or in, a cell in a variety of suitable formats. In some embodiments, the RNA-guided endonuclease is encoded by a plasmid. The plasmid can be replication-competent or replication-incompetent, and is preferably replication-competent. The plasmid can be the same plasmid or a different plasmid than a plasmid encoding a guide RNA and/or a plasmid encoding a donor polynucleotide. In some cases, the RNA-guided endonuclease is encoded by a first plasmid and the guide RNA is encoded by a second plasmid. In some cases,a donor fragment is encoded by the first plasmid. In some cases, a donor fragment is encoded by the second plasmid. In some cases, the donor fragment is encoded by a third plasmid.

Plasmids of the invention can comprise a C. glutamicum and/or E. coli compatible origin of replication. In some cases, the plasmid comprises a all or C ASE I origin. In some cases, the plasmid comprises a colE1, pl5a, or R6k origin. In some cases, the plasmid comprises an origin selected from CG1, and CASE1 and an origin selected from colE1, p15a, and R6k.

As described herein, in some cases one or more of donor fragment, RNA-guided endonuclease, and/or guide RNA is encoded in a linear or circular, non-plasmid, nucleic acid fragment. The one or more fragments can be integrated into the genome. Thus, in some embodiments, the RNA-guided endonuclease can be encoded in a nucleic acid fragment that is integrated into the genome of the cell to be edited. In some cases, the plasmid or integrated fragment further contains a sequence for negative selection (e.g., mazF, ccdB, gala-1, lacY, thyA, pheS, tetAR, rpsL, sacB, a temperature sensitive replication origin and the like) and/or flanking recombination sequences such as FLPs, loxP sequences, or the like, that can be activated at a later time for removal of the RNA-guided endonuclease encoding sequence.

The nucleic acid (e.g., linear or circular fragment or plasmid) encoding the RNA-guided endonuclease can contain a selection marker. Suitable selection markers include, but are not limited to, antibiotic resistance genes such as a chloramphenicol resistance gene, an ampicillin resistance gene, a tetracycline resistance gene, a Zemin resistance gene, a spectinomycin resistance gene and a Km (Kanamycin resistance gene), tetA (tetracycline resistance gene), G418 (neomycin resistance gene), van (vancomycin resistance gene), tet (tetracycline resistance gene), ampicillin (ampicillin resistance gene), methicillin (methicillin resistance gene), penicillin (penicillin resistance gene), oxacillin (oxacillin resistance gene), erythromycin (erythromycin resistance gene), linezolid (linezolid resistance gene), puromycin (puromycin resistance gene) or a hygromycin (hygromycin resistance gene).

In some cases, the selection marker in the RNA-guided endonuclease encoding nucleic acid (e.g., linear or circular fragment or plasmid) is the same selection marker as used in different nucleic acid encoding a guide RNA and/or donor polynucleotide. In some cases, the selection marker in the RNA-guided endonuclease encoding plasmid is a different selection marker as compared to a selection marker in a different nucleic acid encoding a guide RNA and/or donor polynucleotide. The use of one or more positive and/or negative selection markers can allow specific and differential selection for the individual CRISPR components. For example, a cell can be edited by providing in the cell an RNA-guided endonuclease polypeptide, a first guide RNA, and optionally a first donor fragment; and then a second edit can be made by curing the cell of the first guide RNA and donor fragment; and providing into the cell a second guide RNA and/or donor fragment. The RNA-guided endonuclease, guide RNA, and/or donor fragment can be provided into the cell by introducing a nucleic acid encoding the CRISPR component(s), introducing a nucleoprotein complex of one or more CRISPR component(s), inducing expression of one or more CRISPR component(s), or a combination thereof.

In some embodiments, the RNA-guided endonuclease sequence is operably linked to a constitutive promoter. In some embodiments, the RNA-guided endonuclease sequence is operably linked to an inducible promoter. In some embodiments, the RNA-guided endonuclease sequence is operably linked to a native promoter. In some embodiments, the RNA-guided endonuclease sequence is operably linked to an exogenous promoter. In some embodiments, the RNA-guided endonuclease sequence is operably linked to a synthetic promoter.

### Donor Polynueleotides

By a “donor polynucleotide” or “repair fragment” is meant a nucleic acid sequence to be inserted at the cleavage site induced by the RNA-guided endonuclease (e.g, a Cas9 potypeptide). A suitable donor polynucleotide sequence will generally comprise a left homology arm sequence and a right homology arm sequence each homologous to a Corynebacterium target sequence, and will further comprise at least one mutation sequence flanked by the left and right homology arm sequences. In some cases, the donor polynucleotide comprises two or more mutation sequences, wherein at least two, or all, of the two or more mutation sequences are either both flanked by the same left and right homology arm sequences, or at least two, or all, of the two or more mutation sequences are flanked by different left and right homology arm sequences. Generally, where two or more mutation sequences are in the same donor polynucleotide, the mutation sequences are mutations of target genome loci in close proximity to each other. Typically, the two or more mutation sequences on a donor polynucleotide encode genome modifications that are within, or within about, 150 base pairs, 125 base pairs, 100 base pairs, 75 base pairs, 70 base pairs, 65 base pairs, 60 base pairs, 55 base pairs, 50 base pairs, 45 base pairs, 40 base pairs, 35 base pairs, 30 base pairs, 25 base pairs, 20 base pairs, or 10 or 5 base pairs. In sonic cases, the two or more mutation sequences encode genome modifications that are in close proximity to one another in the genome are at a distance from each other in the genome of from about 10 to about 100 base pairs, or from about 25 to about 75 base pairs.

As demonstrated herein,the editing efficiency of the CRISPR/Cas9 complex in Counebacterium increases significantly with increasing homology arm length. Accordingly, in some embodiments, the right and left homology arm sequences used in combination with an RNA-guided endonuclease polypeptide as described herein each independently comprises, comprises about, comprises at least, or comprises at least about, 25; 45, 50; 75; 100; 125; 150; 175; 200; 225; 250; 275; 300; 325; 350; 375; 400; 425; 450; 475; 500; 525; 550; 575; 600; 625; 650 675; 700; 725; 750; 775; 800; 825; 850; 875; 900; 925; 950; 975; 1,000; 1,025; 1,050; 1,075; 1.100; 1,125; 1,150; 1,175; 1,200; 1,225; 1,250; 1,275; 1,300; 1,325; 1,350; 1,375; 1,400; 1,425; 1,450; 1,475; 1,500; 1525; 1,550; 1.575; 1,600; 1.625; 1,650; 1,675 1,700; 1,725; 1,750; 1,775 1,800; 1,825 1,850; 1,875; 1,900; 1,925; 1,950; or 2,000 base pairs. In some embodiments, the left and right homology aim sequences used in combination with an RNA-guided endonuclease polypeptide as described herein each independently comprises no more than, or no more than about, 25; 50; 75; 100; 125; 150; 175; 200; 225; 250; 275; 300; 325; 350; 375; 400; 425; 450; 475; 500; 525; 550; 575; 600; 625; 650; 675; 700; 725; 750; 775; 800; 825; 850; 875; 900; 925; 950; 975; 1,000; 1,025; 1,050; 1,075; 1,100; 1,125; 1,150; 1,175; 1,200; 1,225; 1,250; 1,275; 1,300; 1,325; 1,350; 1,375; 1,400; 1,425; 1,450; 1,475; 1,500; 1;525; 1,550; 1,575; 1,600; 1,625; 1,650; 1,675; 1,700; 1,725; 1,750; 1,775; 1,800; 1,825; 1,850; 1,875; 1,900; 1,925; 1,950; or 2,000 base pairs.

In certain embodiments, the right and left homology arm sequences used in combination with an RNA-guided endonuclease polypeptide as described herein each independently comprise between about 45 and about 125 base pairs, between about 25 and about 2000 base pairs, between about 25 and about 1000 base pairs, between about 25 and about 600 base pairs, between about 25 and about 500 base pairs, between about 25 and about 250 base pairs, between about 25 and about 200 base pairs, between about 25 and about 100 base pairs, or between about 25 and about 50 base pairs. In certain embodiments, the right and left homology arm sequences used in combination with an RNA-guided endonuclease polypeptide as described herein each independently comprise between about 100 and about 2000 base pairs, between about 100 and about 1000 base pairs, between about 100 and about 600 base pairs, between about 100 and about 500 base pairs, between about 100 and about 250 base pairs, between about 100 and about 200 base pairs, or between about 100 and about 150 base pairs. In certain embodiments, the right and left homology arm sequences used in combination with an RNA-guided endonuclease polypeptide as described herein each independently comprise between about 0 and about 2000 base pairs, between about 0 and about 1000 base pairs, between about 0 and about 600 base pairs, between about 0 and about 500 base pairs, between about 0 and about 250 base pairs, between about 0 and about 200 base pairs, between about 0 and about 100 base pairs, between about 0 and about 50 base pairs, or between about 0 and about 25 base pairs,

In some cases, the right homology arm used in combination with an RNA-guided endonuclease polypeptide as described herein has a length of 0 base pairs, while the left homology arm has a length of, of at least, of about, or of at least about 25, 45, 50, 75, 100, 150, 175, 200, 225, 250, 275, 300, 325, 350, 375, 400, 425, 450, 475, 500, 525, 550, 575, 600, 625, 650, 675, 700, 725. 750, 775, 800, 825, 850, 875, 900, 925, 950, 975, 1000, 1025. 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625. 1650, 1675, 1700, 1725, 1750, 1775, 1800, 1825, 1850, 1875, 1900, 1925, 1950, or 2000 base pairs. In some cases, the left homology arm used in combination with an RNA-guided endonuclease polypeptide as described herein has a length of 0 base pairs, while the right homology arm has a length of, of at least, of about, or of at least about 25, 45, 50, 75, 100, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, or 2000 base pairs.

The donor polynucleotide i s typically not identical to the genomic sequence that it replaces. Rather, the donor polynucleotide generally comprises at least one mutation sequence, e.g., one or more single base changes, insertions, deletions, inversions or rearrangements with respect to the genomic sequence, so long as sufficient homology is present to support homology-directed repair. Exemplary mutation sequences include: a single nucleotide insertion; an insertion of two or more nucleotides; an insertion of a nucleic acid sequence encoding one or more proteins; a single nucleotide deletion; a deletion of two or more nucleotides; a deletion of one or more coding sequences; a substitution of a single nucleotide; a substitution of two or more nucleotides; two or more non-contiguous insertions, deletions, and/or substitutions; or any combination thereof. In a specific embodiment, the at least one mutation sequence comprises a mutation of a Cas9 PAM.

In some embodiments, the donor polynucleotide comprises a mutation sequence having two or more non-contiguous mutations. For example, the donor polynucleotide can comprise a mutation in an RNA-guided endonuclease polypeptide PAM region (e.g., Cas9 PAM region), optionally or alternatively a mutation in an RNA-guided endonuclease polypeptide seed region (e.g., Cas9 seed region), and a mutation at least 5, 10, 15, 20, 25, 30, 45, 50, 60, 90, or 100 nucleotides away. In some cases, the non-contiguous modifications that are in close proximity to one another in the genome are within, or within about, 200 base pairs, 175 base pairs, 150 base pairs, 125 base pairs, 100 base pairs, 75 base pairs, 70 base pairs, 65 base pairs, 60 base pairs, 55 base pairs, 50 base pairs, 45 base pairs, 40 base pairs. 35 base pairs, 30 base pairs, 25 base pairs, 20 base pairs, or 10 base pairs, or 5 base pairs. In some cases, the non-contiguous modifications that are in close proximity to one another in the genome are at a distance from each other in the genome of from about 10 to about 100 base pairs, or from about 25 to about 75 base pairs.

In some cases one donor polynucleotide comprises two or more non-contiguous mutations and a second or other donor polynucleotide comprises a mutation at a different locus. In some cases one donor polynucleotide comprises two or more non-contiguous sequences and a second or other donor polynucleotide comprises two or more non-contiguous mutations at a different locus.

The mutation sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some embodiments may be used for other purposes (e.g., to signify expression at the targeted genomic locus). In some embodiments, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor polynucleotide may be provided as a single-stranded DNA, or double-stranded DNA. The ends of the donor polynucleotide may be protected (e.g., from exonucleolytic degradation) by methods known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s), phosphate groups, methyl groups, and the use of modified internucleotide linkages such as, for example, phosphorothioates, phosphoramidates, and O-methyl ribose or deoxyribose residues.

In some embodiments, the donor polynucleotide is provided, e.g., introduced into a cell, as part of a plasmid having additional sequences such as, for example, a replication origin, one or more promoters, and/or positive or negative selection markers, or a combination of two thereof, three thereof, or all thereof. In some embodiments, the donor polynucleotide is provided as part of a replication-competent plasmid. In some embodiments, the donor polynucleotide is introduced into a cell as part of a replication-incompetent plasmid. Alternatively, donor polynucleotides can be introduced as naked nucleic acid (e.g., as a linear or circular fragment), as nucleic acid complexed with an agent such as a liposome or polymer, or can be delivered by viruses (e.g., adenovirus, AAV).

In some embodiments, incorporation of the donor polynucleotide can be aided by the simultaneous or sequential introduction of recombination proteins such as RecE/T, or one or more components of the phage lambda-derived Red recombination system lambda exonuclease, beta-protein, and/or gamma-protein (See, GENETICS Nov. 1, 2010 vol. 186 no. 3 791-799),

In certain embodiments, two or more donor fragment-encoding nucleic acids are operably linked to differentially inducible promoters for selective induction, such as for serial editing of a host cell genome.

Without wishing to be bound by theory, the present inventors hypothesize that multiplexed genome editing with plasmid-based presentation of donor polynucleotides can proceed via one or more of the mechanisms (1), (2), (3), (4), or (5) detailed below.

Mechanism (1), single crossover loop-in followed by RNA-guided endonuclease polypeptide (e.g., Cas9)/sgRNA-mediated cut, and repair. Within the cell RNA-guided endonuclease polypeptide Cas9) is constitutively expressed. Upon transformation of the sgRNA/repair fragment construct, there is a loop-in event at the repair fragment loci (i.e., two separate integration events), thereby duplicating the loci (i.e., one mutant copy, one wild-type copy). In parallel, the sgRNA.(s) on the construct are expressed, fold, and bind to RNA-guided endonuclease polypeptide (e.g., Cas9)-priming it for target recognition and cutting. At this point, “primed RNA-guided endonuclease polypeptide (e.g., Cas9)” recognizes and cleaves the wild-type locus. The cell must repair this break in order to survive. The mutant locus, already integrated into the genome, is adjacent to the cut site and serves as a recombination template for repair. The mutation then becomes fixed in the genomic DNA and persist to daughter cells.

Mechanism (2), double crossover loop-in/loop-out followed by RNA-guided endonuclease polypeptide (e.g., Cas9)/sgRNA-mediated cleavage. Within the cell RNA-guided endonuclease polypeptide (e.g., Cas9) is constitutively expressed. Upon transformation of the sgRNA/repair fragment construct, there is a loop-in event at the repair fragment loci (i.e., two separate integration events), thereby duplicating the loci (i.e., one mutant copy, one wild-type copy). Following loop-in of the sgRNA/repair fragment construct, there is a loop-out event mediated by the repair fragment homology arms. (In theory, ˜50% of the cells should loop-out to the wild-type version of the locus, the other 50% will loop-out to the mutant version of the locus.) In parallel, the sgRNA(s) on the construct are expressed, fold, and bind to RNA-guided endonuclease polypeptide (e.g., Cas9)- priming it for target recognition and cutting. At this point, “primed RNA-guided endonuclease polypeptide (e.g., Cas9)” recognizes and cleaves cells that have looped-out to wild-type, clearing them from the population. Cells containing the mutant locus are not cleaved by “primed RNA-guided endonuclease polypeptide (e.g., Cas9)”; the mutation then becomes fixed in the genomic DNA and persist to daughter cells.

Mechanism (3), RNA-guided endonuclease polypeptide (e.g., Cas9)/sgRNA-mediated cut followed by double crossover repair with plasmid donors. Within the cell Cas9 is constitutively expressed. Upon transformation of the sgRNAlrepair fragment construct, the sgRNA(s) on the construct are expressed, fold, and bind to RNA-guided endonuclease polypeptide Cas9)-priming it for target recognition and cutting. At this point, “primed RNA-guided endonuclease polypeptide (e.g., Cas9)” recognizes and cleaves the wild-type loci (i.e., two double-stranded breaks in the chromosomal DNA). The cell must repair these breaks in order to survive. The sgRNA/repair fragment construct serves as a recombinational template to fix the breaks in the DNA (i.e., two double crossover repair events). The cell performs the double crossover events and repairs the breaks. The mutations become fixed in the genomic DNA and persist to daughter cells.

Mechanism (4), RNA-guided endonuclease polypeptide (e.g., Cas9)/sgRNA-mediated cut followed by double crossover repair with double-stranded linear donors: Within the cell RNA-guided endonuclease polypeptide (e.g., Cas9) is constitutively expressed along with heterologous recombination proteins (e.g., beta, gam, and exo from lambda red recombination system or RecE/RecT from the rac prophage). Upon transformation of the sgRNA construct and linear double-stranded repair fragments), the sgRNA(s) on the construct are expressed, fold, and bind to RNA-guided endonuclease polypeptide (e.g., Cas9)-priming it for target recognition and cutting. At this point, “primed Cas9” recognizes and cleaves the wild-type loci (i.e., two double-stranded breaks in the chromosomal DNA). The cell must repair these breaks in order to survive. The repair fragments are processed by the heterologous recombination proteins and are used as templates for chromosomal repair. The mutations become fixed in the genomic DNA and persist to daughter cells.

Mechanism (5), introduction of single-stranded linear donors via DNA replication followed by RNA-guided endonuclease polypeptide (e.g., Cas9)/sgRNA-mediated cleavage of wild-type loci. Within the cell RNA-guided endonuclease polypeptide (e.g., Cas9) is constitutively expressed along with a heterologous recombination protein (e.g., gam from lambda red recombination system or RecT from the rac prophage). Upon transformation of the sgRNA construct and linear single-stranded repair fragment(s), the linear single-stranded repair fragment(s) are incorporated into the genomic DNA via Okazaki fragment extension during DNA replication. The sgRNA.(; s) are expressed, fold, and bind to RNA-guided endonuclease polypeptide (e.g., Cas9)-priming, it for target recognition and cutting. At this point, “primed RNA-guided endonuclease polypeptide (e.g., Cas9)” recognizes and cleaves the wild-type loci (i.e., two double-stranded breaks in the chromosomal DNA), leaving the altered loci intact. The mutations become fixed in the genomic DNA and persist to daughter cells.

### Guide RNAs

The guide RNA may be provided as: double-stranded DNA encoding the guide RNA, single-stranded RNA, or double-stranded RNA. In some embodiments, the guide RNA is encoded. in a plasmid having additional sequences such as, for example, a replication origin, one or more promoters, and/or positive or negative selection markers, or a combination of two thereof, three thereof, or all thereof. In some embodiments, the guide RNA is provided as part of a replication-competent plasmid. In some embodiments, the guide RNA is provided as part of a replication-incompetent plasmid. Alternatively, guide RNAs can be provided as naked nucleic acid (e.g., as a linear or circular fragment), as nucleic acid complexed with an agent such as a liposome or polymer, or can be delivered by viruses (e.g., adenovirus, AAV).

In certain embodiments, two or more guide RNA-encoding nucleic acids are operably linked to differentially inducible promoters for selective induction, such as for serial editing of a host cell genome. The differentially inducible guide RNAs can be encoded by the same or a different pla.smid or nucleic acid fragment.

### DNA Repair Components

In certain embodiments, methods are provided for editing a host cell genome with an RNA-guided endonuclease, a donor polynucleotide, and a nucleic acid encoding a component of a heterologous DNA repair pathway. In some cases, the method includes editing a host cell genome with an RNA-guided endonuclease, a donor polynucleotide, and two or more nucleic acids encoding two or more components of a heterologous DNA repair pathway. In some cases, the method includes an RNA-guided endonuclease, a donor polynucleotide, and a nucleic acid encoding two or more components of a heterologous DNA repair pathway.

In some cases, the repair pathway is a RecA/RecBCD repair pathway. In some cases, the repair pathway is a RecE/RecT repair pathway. In some cases, the repair pathway is a RedalRediβ repair pathway. In some cases, the repair pathway is a lambda-derived red recombination repair pathway. In some cases, the method includes expression of RecA and/or RecBCD. In some cases, the method includes expression of RecE and/or RecT. In some cases, the method includes expression of Redα and/or Redβ. In some cases, the method includes expression of beta, gam, and/or exo components of the lambda-derived red recombination repair pathway. A nucleic acid encoding a component of the heterologous DNA repair pathway can be on a first, second, or other plasmid. In some cases, sgRNA(s) are encoded on a first plasmid and heterologous DNA repair protein(s) are encoded on a second plasmid.

### Expression, Purification, and Delivery

In one aspect, the present disclosure provides plasmids, vectors, constructs, and nucleic acid sequences encoding the CRISPR/RNA-guided endonuclease polypeptide (e.g, Cas9) gene editing complexes. In certain embodiments, the present disclosure provides plasmids for transient expression of the guide RNA, with or without simultaneous or sequential expression of the RNA-guided endonuclease (e.g., Cas9) polypeptide and/or presentation of the donor polynucleotide. In some embodiments the plasmids and vectors of the present invention will encode the guide RNA and also encode the RNA-guided endonuclease (e.g., Cas9) polypeptide and/or donor polynucleotide of the present disclosure. In other aspects, the different components of the engineered complex can be encoded in one or more distinct plasmids.

In some embodiments, the plasmids of the present disclosure can be used across multiple Corynebacterium species. In some embodiments, the plasmids of the present disclosure are tailored specifically to C. glutamicum. In some embodiments, the plasmids of the present disclosure are, or contain sequences (e.g., promoter, guide RNA, RNA-guided endonuclease polypeptide (e.g., Cas gene), replication origin, etc.) that are, codon-optimized to express in Cognebacterium in general, and/or C. glutamicum in particular, and/or a specific strain thereof, such as C. glutamicum NRRL-B 11474.

In some embodiments, the plasmids and vectors of the present disclosure are selectively expressed in the cells of interest. Thus, in some embodiments, the present application contemplates the use of ectopic promoters, developmentally-regulated promoters, and/or inducible promoters. In some embodiments, the present disclosure provides the use of terminator sequences.

### Transformation

In some embodiments, the present specification provides the use of transformation of the plasmids and vectors disclosed herein. Persons having skill in the art will recognize that the plasmids of the present specification can be transformed into cells through any known system as described in other portions of this specification. For example, in sonic aspects, the present specification provides transformation by electroporation, chemically-induced transformation (e.g., transformation in the presence of a divalent cation such as Mg²⁺), conjugation, particle bombardment, agrobacterium transformation, nano-spike tra.nsformation, and virus transformation (e.g., phage transformation).

In some embodiments, the vectors of the present specification may be introduced into the Corynebacterium host cells using any of a variety of techniques, including transformation, transfection, transduction, viral infection, gene guns, or Ti-mediated gene transfer. Particular methods include calcium phosphate transfection, DEAE.-Dextran mediated transfection, lipofection, or electroporation (Davis et al., 1986 “Basic Methods in Molecular Biology”; Van der Rest et al, Appl Microbiol Biotechnol. 1999 Octobter; 52(4):541-5). Other methods of transformation include, e.g., lithium acetate transformation and electroporation. See, e.g., Gietz et al., Nucleic Acids Res. 27:69-74 (1992); Ito et al., J. Bacterol. 153:163-168 (1983); and Becker and Guarente, Methods in Enzymology 194:182-187 (1991). In some embodiments, transformed host cells are referred to as recombinant Corynebacterium host strains.

In some embodiments, the present specification provides high-throughput transformation of cells using 96-well plate robotics platform and liquid handling machines, as described in PCT/US2017/040114, entitled Apparatuses and methods for electroporation.

In some embodiments, methods for introducing exogenous protein (e.g. RNA-guided endonuclease (e.g., Cas9) polypeptides) into cells are required. Various methods for achieving this have been described previously including direct transfection of protein/RNA/DNA or DNA transformation followed by intracellular expression of RNA and protein (See, e.g., Dicarlo et al., Nucleic Acids Res 41:4336-43 (2013); Ren et al., Gene 195:303-311 (1997); Lin et al. Elife 3:e04766 (2014)).

In some embodiments, the present specification provides screening transformed cells with one or more selection markers as described above. In one such embodiment, cells transformed with a vector comprising a kanamycin resistance marker (KanR) are plated on media containing effective amounts of the kanamycin antibiotic. Colony forming units visible on kanamycin-laced media are presumed to have incorporated the vector cassette into their genome. Insertion of the desired sequences can be confirmed via PCR, restriction enzyme analysis, and/or sequencing of the relevant insertion site.

Persons having skill in the art will readily recognize that viral vectors or plasmids for gene expression can be used to deliver the sequences and/or complexes disclosed herein. Virus-like particles (VLP) can be used to encapsulate nucleic acids or nucleoprotein complexes for recombinant expression, or purified ribonucleoprotein complexes disclosed herein can be provided and delivered to cells via electroporation, contacting cell(s) with VLP, or injection.

### Kits

In some embodiments, the disclosure provides kits containing any one or more of the elements disclosed in the above methods and compositions. In some aspects, the kit comprises a CRISPR/RNA-guided endonuclease polypeptide (e.g., Cas9) system and instructions for using the kit. In some aspects, the CRISPR/RNA-guided endonuclease polypeptide (e.g., Cas9)system comprises a plasmid comprising a promoter operably linked to a sequence for expressing a first guide RNA, and a first donor polynucleotide having an upstream homology arm sequence and a downstream homology arm sequence each homologous to a Corynebacterium target sequence, said first donor polynucleotide including at least one mutation sequence flanked by said upstream homology arm sequence and said downstream homology arm sequence, and optionally a RNA-guided endonuclease (e.g., Cas9)polypeptide, which may also be directly integrated into the host Corynebacterium strain. The donor polynucleotide and/or RNA-guided endonuclease (e.g., Cas9) polypeptide may be encoded on the same or separate plasmids as the guide RNA. Alternatively, the donor polynucleotide may be provided as a linear or circular fragment.

Elements may be provided individually or in combinations, and may be provided in any suitable container, such as a vial, a bottle, a tube, or a multi-well plate (e.g., 96-well, 384-well, or 1536-well plate). In some aspects, the kit includes instructions in one or more languages, for example in more than one language.

In some aspects, a kit comprises one or more reagents for use in a process utilizing one or more of the elements described herein (e.g., purified RNA-guided endonuclease (e.g., Cas9) polypeptide). Reagents may be provided in any suitable container. For example, a kit may provide one or more reaction or storage buffers. Reagents may be provided in a form that is usable in a particular assay, or in a form that requires addition of one or more other components before use (e.g., in concentrated or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some aspects, the buffer is alkaline. In some aspects, the buffer has a pH from about 7 to about 10. In some aspects, the kit comprises one or more oligonucleotides corresponding to a crRNA sequence for insertion into a vector so as to operably link the crRNA sequence and a regulatory element.

Having now generally described the invention, the same will be more readily understood through reference to the following examples that are provided by way of illustration, and are not intended to be limiting of the present invention, unless specified.

Each periodical, patent, and other document or reference cited herein is herein incorporated by reference in its entirety.

### EXAMPLES

### Example 1

### Cas9 Can Induce Lethal DSBs in C. glutamicum when Expressed in Conjunction with Functional Guide RNA

The Cas9 gene from Streptococcus pyogenes with a codon bias for Streptomyces (Cobb et al. ACS Synth. Biol. 4, 723-728 (2015)) was synthesized and linked to the Ptrc promoter and integrated into NRRL-1311474 Corynebacterium glutamicum for expression of Cas9.

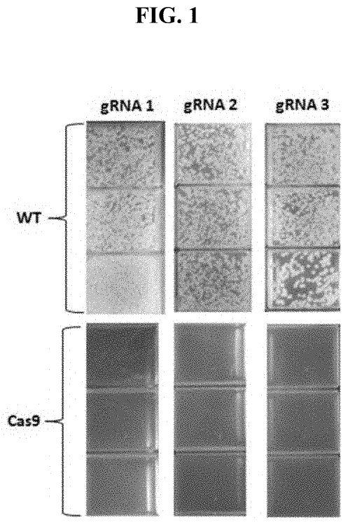

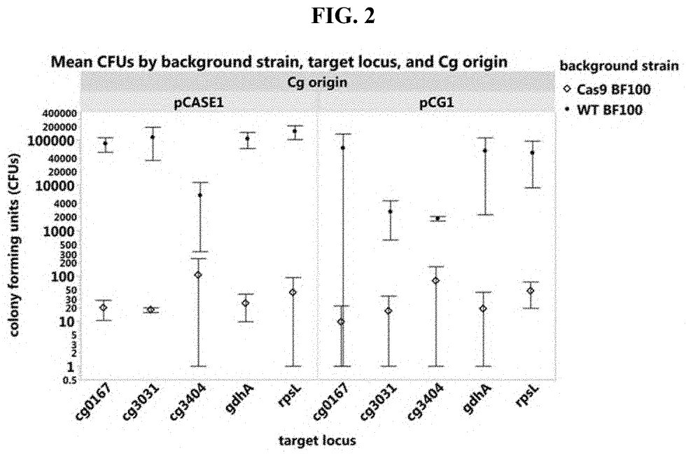

Cas9 activity was tested in a strain where Cas9 was integrated in the cg0443-cg0444 locus. As double stranded breaks (DSBs) are lethal when repair is ineffective, no colonies were expected to form beyond a few escape mutants (FIG. 1). Upon transformation of plasmids with Peg2613 as the promoter driving guide RNA expression, as shown in FIG. 1, the lethal effect of the resulting Cas9 DSB was demonstrated and thus Cas9 was functional in NRRL-B11474 C. glutamicum. FIG. 2 demonstrates that the lethal effect of a functioning sgRNA can be generalized across a variety of loci of interest.

### Example 2

### CRISPR/Cas9 Genome Editing-SNP Introduction

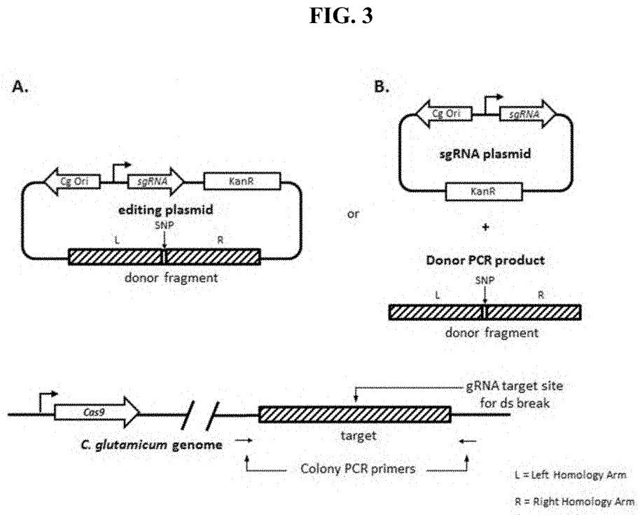

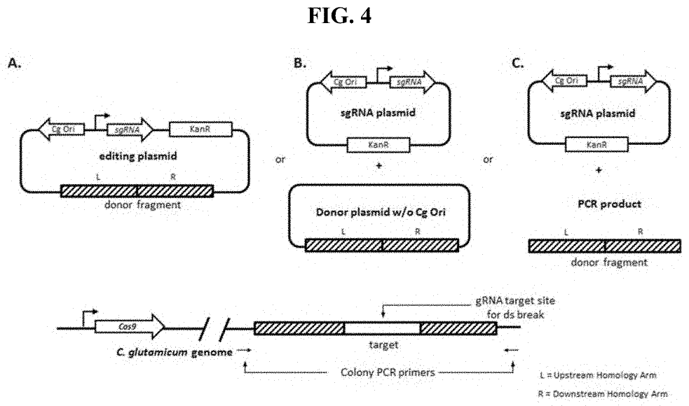

After successfully demonstrating the functionality of Cas9 and the guide RNAs to be used, plasmids were designed to introduce SNPs at 3 test loci using the validated guide RNAs and a corresponding donor polynucleotide encoded together on a single plasmid. A schematic of the configuration used to introduce SNPs is shown in Panel A of FIG. 3. Targeted SNPs were single mutations in each locus that alter the PAM region. Target SNPs at the PAM region prevent subsequent cutting of the modified genome by the CRISPR/Cas9 complex. The results were compared to a strain containing Cas9 and expressing only guide RNA, and NRRL-B11474 C. glutamicum without Cas9 integrated but with plasmids containing identical guide RNA and donor fragments used in Cas9 integrated strains.

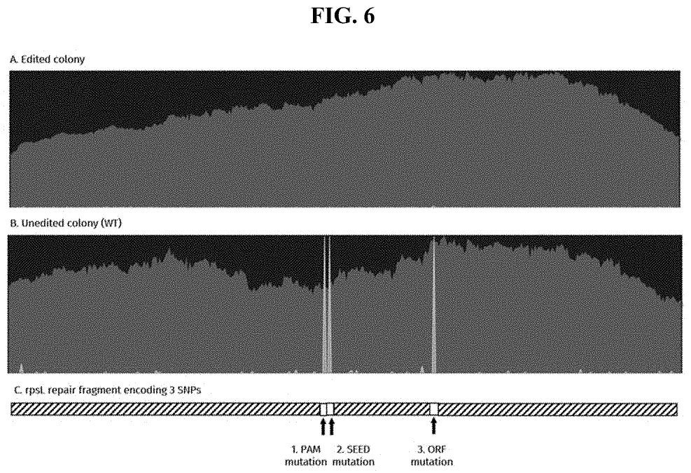

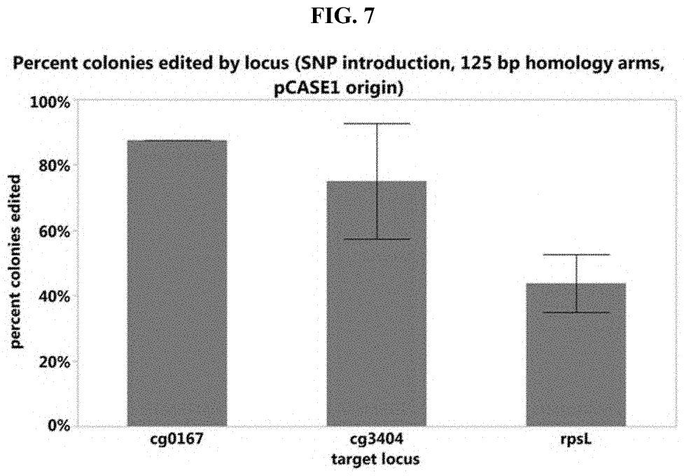

Colonies from a transformation with the guide RNA/donor DNA plasmid were tested via colony PCR and NGS sequence analysis. An example of one NGS coverage plot is depicted in FIG. 6. Three loci were selected to test SNP introduction (rpsL, cg0167 and cg3404). The percent of colonies transformed with the guide RNAIdonor DNA plasmid that contained the PAM site mutation was 44% (rpsL), 87.5% (cg0167) and 75% (cg3404) (FIG. 7). Test loci range across the genome and represent a variety of genes of known and unknown function, indicating this method of genome editing is generalizable to a wide range of gene targets.

### Example 3

### CRISPR/Cas9 Genome Editing-Gene Deletion

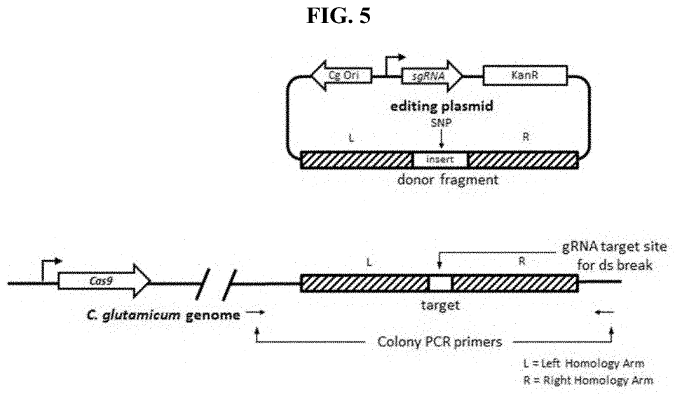

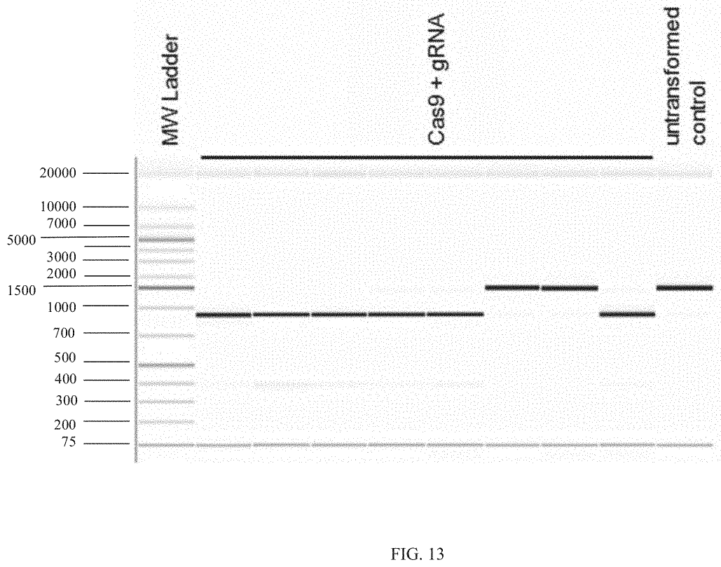

Deletion of 702 by from the cg3031 locus was tested. An overview of the strategy to knock out the cg3031 ORF in C. glutamicum is provided in Panel A of FIG. 4. As in the above examples Cas9 was integrated into the genome; donor polynucleotides containing 340 by left arm homology and 400 bp right arm homology to the cg3031 ORF and a guide RNA cassette were introduced on a single plasmid. Removal of the 702 bp region of cg3031 was detectable by PCR, as shown in FIG. 13. A 1648 by band is indicative of a wild-type genome; while a 946 bp band is indicative of a modified genome. Analysis of colonies produced after transformation demonstrated the presence of the deletion of 702 bp from the cg3031 locus in 6 out of 8 colonies.

### Example 4

### CRISPR/Cas9 Genome Editing-Small Insertions

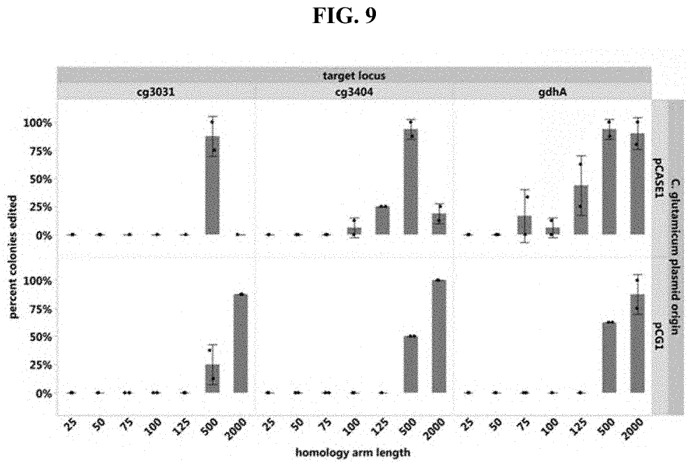

Polynucleotides were designed to insert 100 bp at three loci as illustrated in FIG. 5. Each insertion was designed to delete 20 bp of the protospacer targeted and replace with a 100 bp insertion. Insertions were designed to target three loci (gdhA, cg3031 and cg3404). Donor fragments contained homology ann lengths of 25, 50, 75, 100, and 125, 500 and 2000 bp. Polynucleotides with each donor fragment were transformed into NRRL-B11474 C. glutamicum with integrated Cas9. All three loci were successfully edited with insertions (FIG. 9). Homology arms with 500 bp were able to create insertions at all three test loci while lower homology arms showed variability across loci (FIG. 9).

### Example 5

### CRISPR/Cas9 Genome Editing-Successful Simultaneous Introduction of Multiple Co-Located SNPs at Multiple Loci

If a target SNP is positioned outside of a PAM region or if multiple SNPs are desirable then multiple co-located SNPs can be introduced on the same donor fragment. To explore the simultaneous introduction of multiple co-located SNPs, donor fragments were designed to introduce two simultaneous SNPs at the cg0167 and cg3404 test loci, and three simultaneous edits at the rpsiL test locus. The donor fragment targeting cg0167 consists of 1 SNP that scrambles the PAM region and another SNP 10 bp away from the PAM. The donor fragment targeting cg3404 includes 1 SNP that scrambles the PAM region and another SNP 70 bp away from the PAM. The rpsL donor fragment includes 1 SNP that scrambles the PAM region, another SNP in the seed region of the protospacer (10 bp downstream of the PAM), and another SNP 65 bp away from the PAM. Target SNPs at the PAM and seed region prevent further cutting of the modified genome by the CRISPR/Cas9 complex. Coverage plots from sequence analysis of edited and unedited colonies are shown in FIG. 6 and demonstrate the successful co-introduction of 3 SNPs at the rpsL locus. Multiple SNPs were successfully introduced at all three loci (FIG. 7).

### Example 6

### CRISPR/Cas9 Editing Efficiency Varies Depending on Length of Homology Arms in Plasmid-Encoded Donor Polynucleotide

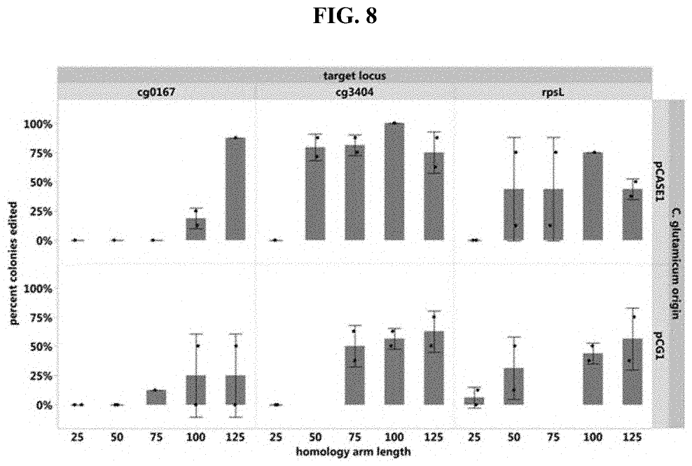

Targeted SNPs and insertions were tested at three loci with different length homology arms. Donor fragments contained left and right symmetrical homology arm lengths of 25, 50, 75, 100, and 125 bp. Target. SNPs were generated at three test loci (cg0167, cg3404, and Ips.11,) and longer homology arms resulted in higher percentages of colonies edited (FIG. 8). SNP editing was demonstrated with homology arms as small as 25 bp at 1 locus (rpsL). Insertions were also tested at an alternative set of three loci (cg3031, cg3404, and gdhA),Longer homology arms resulted in higher percentages of colonies edited (FIG. 9). The smallest homology arm length that resulted in a successful insertion at 1 locus (gdh4) was 75 bp.

### Example 7

### Transformation Efficiency Depends on Origin of Replication and is Unique in NRRL-B114174 Strain of C. glutamicum

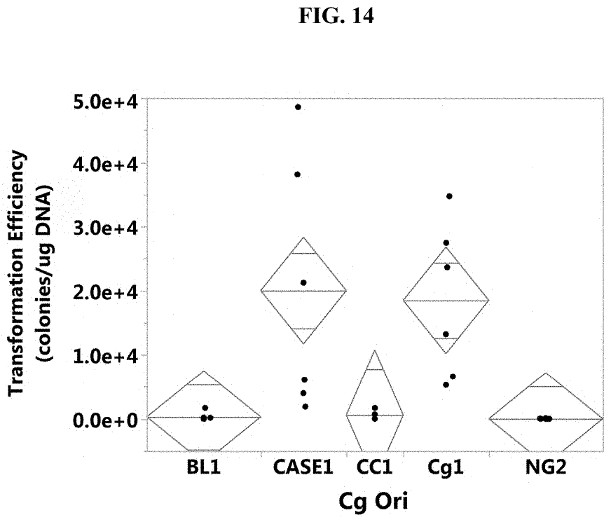

A panel of five C. glutamicum origins of replication were built into plasmids and transformed into Wf NRRE-B11474 C. glutamicum to test transformation efficiency (FIG. 14). Replication origins pCASEI and pCG1 resulted in high numbers of colonies and acceptable transformation efficiency. Origins pBL1, pCC1, & pNG2 resulted in very few colonies and negligible transformation efficiency. The difference in transformation efficiency of pCASE1 & pCG1 in comparison with p131-1, pCC1, & pNG2 is statistically significant. These results stand in contrast to reported use of the latter origins of replication in some strains of C. glutamicum. The data presented herein suggests that various origins of replication exhibit different transformation efficiencies in different industrially-relevant strains of C. glutamicum. Origins pCASE1 and pCG1 were advanced for further work investigating the impact of plasmid origin of replication on editing efficiency in NRRL-B11474. Editing efficiency is expected to be negligible for origins pBL1, pCC1, & pNG2 because successful transformants are a prerequisite of successful editing in this system.

### Example 8

### Origin of Replication Impacts Editing Efficiency

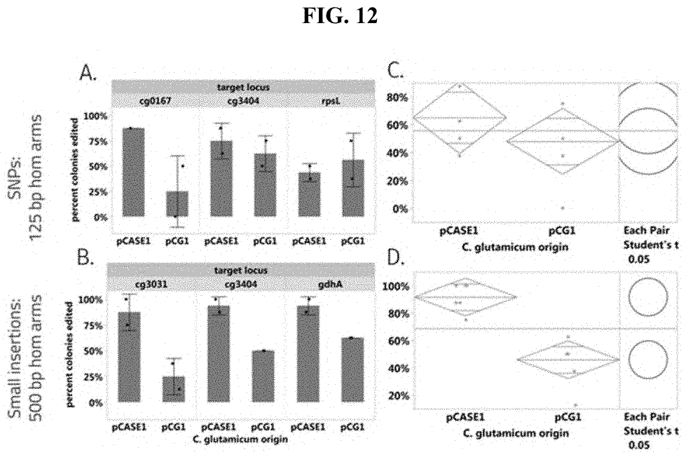

Polynucleotide copy number may impact expression levels of guide RNA and delivery of donor fragments. To investigate if origin of replication has an impact on editing efficiency two C. glutamicum origins of replication (pCASE1 and pCG1) were included in polynucleotides containing guide RNA specific to the target locus, and a donor fragment that contains either 125 bp of homology on either side of the SNP, or 500 bp on either side of the insertion. Plasmids were transformed into a C. glutamicum NRRL-B11474 strain carrying an integrated, constitutively expressed copy of the Cas9 gene, and up to 8 colonies were picked for screening by NGS. Two biological replicates were averaged for each editing construct. Origin of replication had a significant impact on editing efficiency with pCASE1 showing significantly higher editing efficiency than pCG1 (FIG. 12).

### Example 9

### Expression of RecET in Conjunction with PCR Donor Polynucleotide Results in Successful Incorporation of Desired Edits

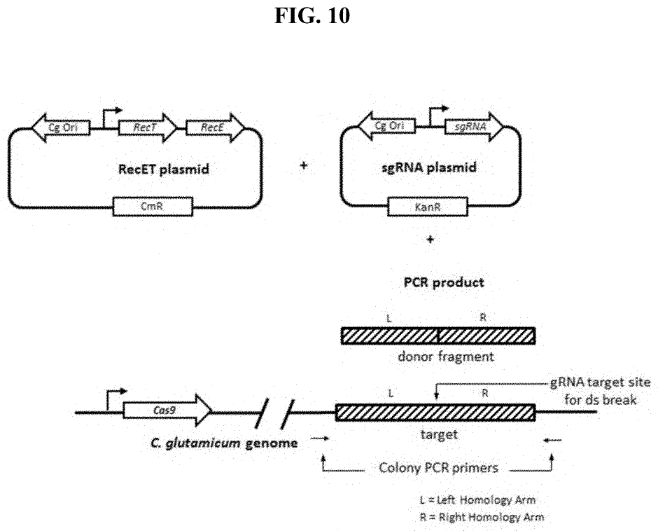

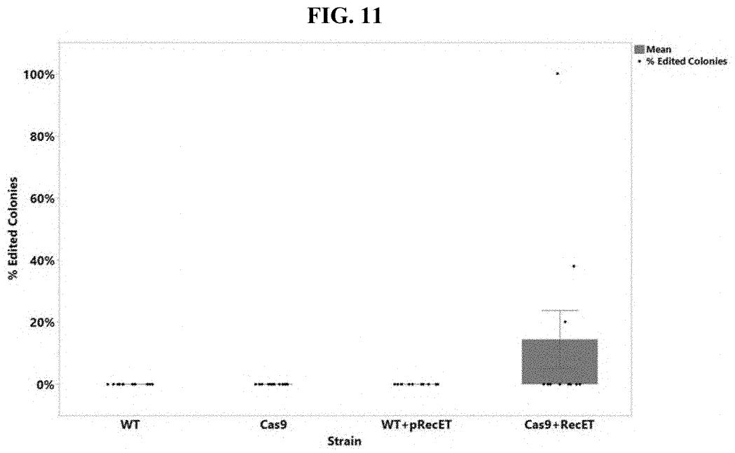

A configuration that can be used to generate edits includes delivery of a guide RNA on a replicating plasmid and a donor fragment as a PCR product. These components were transformed into a strain background containing a helper plasmid containing an inducible promoter operably linked to RecET (pRecET) (FIG. 10). PCR products were designed to create two SNPs at cg3404. PCR donors and guide RNA plasmids were transformed into WT, integrated Cas9, WT containing pRecET, and integrated Cas9 containing pRecET. Colonies were screened via colony PCR and Sanger sequenced to determine if SNPs or knockouts were successfully created. SNPs and knockouts were successfully generated only in the strain with integrated Cas9 and pRecET (FIG. 11).

### Example 10

### Multiplexed Parallel SNP Editing At cg3404 and rpsL Using Plasmid-based Donor Polynucleotides

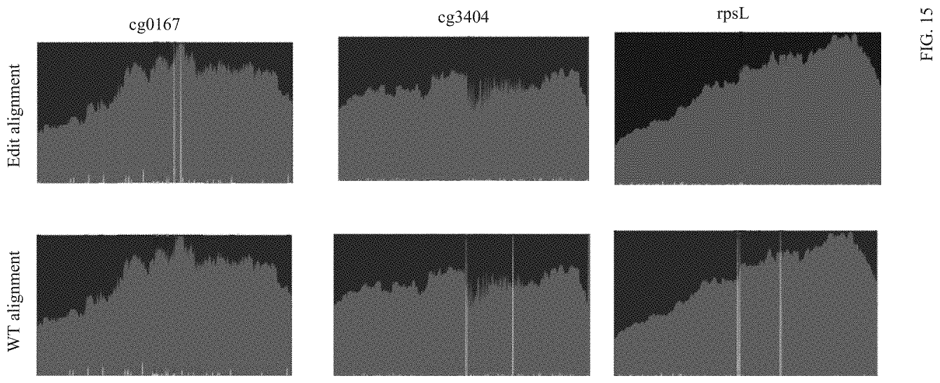

Prior reports suggest that introducing multiple CRISPR Cas9-mediated edits in parallel is an inefficient process. In one experiment (FIG. 15), 2 paired sgRNA & donor fragments were included on a single plasmid that was transformed into a strain carrying an integrated Cas9 gene. Of the colonies screened, simultaneous editing at rpsL and cg3404 from one of the 2-edit constructs in the experiment was observed (as depicted by NGS reads in FIG. 15). In another configuration, a single plasmid containing multiple sgRNAidonor fragment pairs under the control of different inducible promoters can be transformed into a Cas9-expressing strain. Each successive edit can then be introduced by serially inducing the expression of each successive sgRNA. In yet another configuration, the parent strain can be transformed with a plasmid containing multiple repair fragments and not the corresponding gRNAs. Transformants containing the repair fragments could then be transformed with a plasmid containing gRNA(s) corresponding to the already present repair fragments.

### Example 11

### Stacking Genomic Edits by Iterative CRISPR Eediting

Prior reports and our data suggest that introducing multiple edits is inefficient]] that introducing multiple CRISPR Cas9-mediated edits in parallel is an inefficient process.

One alternative is to incorporate multiple edits sequentially. In one such configuration, a plasmid with a single sgRNA/donor fragment pair and containing an element for plasmid clearance can be introduced into the Cas9-expressing, strain. Following transformation and editing, the plasmid can be cleared, and a second plasmid containing a different sgRNA/donor fragment pair can be transformed to introduce a second edit. Colonies can then be assayed to verify the incorporation of all intended edits.

While the present disclosure has been described with reference to preferred embodiments, it will be understood by those skilled in the art that various changes may be made and equivalents may be substituted for elements thereof to adapt to particular situations without departing from the scope of the present disclosure. Therefore, it is intended that the present disclosure not be limited to the particular embodiments disclosed as the best mode contemplated for carrying out the present disclosure, but that the present disclosure will include all embodiments falling within the scope and spirit of the appended claims.

1-49. (canceled)

50. A Counebacterium host comprising:
a first plasmid, wherein said first plasmid comprises a first promoter operably linked to a first guide RNA, and a first donor polynucleotide having at least one mutation sequence flanked by;
a right homology arm sequence and a left homology arm sequence, wherein each homology arm sequence is homologous to a target sequence in a Corynebacterium genome; and
wherein said host has an RNA-guided DNA endonuclease integrated into its genome, wherein said RNA-guided DNA endonuclease is operably linked to an inducible promoter, and comprises a sequence for negative selection and/or flanking recombination sequences.

51-52. (canceled)

53. The host of claim 50, wherein said Corynebacterium host is Corynebacterium glutamicum strain NRRL-B11474.

54. The host of claim 50, wherein the RNA-guided DNA endonuclease is selected from the group consisting of Cas9, Cas12a, Cas12b, Cas12c, Cas12d, Cas12e, Cas12h, Cas13a, Cas13b, Cas13c, Cpf1, and MAD7, or homologs, orthologs, or paralogs thereof.

55-57. (canceled)

58. The host of claim 50, wherein the first plasmid comprises a replication origin selected from the group consisting of a pCASE1 replication origin and a pCG replication origin.

59-67. (canceled)

68. The host of claim 50, wherein said at least one mutation sequence comprises a mutation of an RNA-guided DNA endonuclease protospacer-adjacent motif (PAM) or seed region.

69-73. (canceled)

74. The host of claim 50, wherein said first promoter is Pcg2613.

75-80. (canceled)

81. The host of claim 50, wherein the host comprises a set of proteins from a lambda red recombination system, a Rec ET recombination system, any homologs, orthologs or paralogy of proteins from a lambda red recombination system or a Rec ET recombination system, or any combination thereof.

82. The host of claim 50, wherein the RNA-guided DNA endonuclease is differentially inducible as compared to an inducible promoter operably linked to a guide-RNA.

83. The host of claim 50, wherein the RNA-guided DNA endonuclease is Cas9.

84. The host of claim 83, wherein the Cas9 polypeptide encoding sequence comprises a coding sequence optimized for expression in a Corynebacterium species.

85. The host of claim 50, wherein the recombination sequences are recognized by the recombinase flippase.

86. The host of claim 50, wherein the recombination sequences are recognized by Cre recombinase.
